(19) United States
(12) Patent Application Publication
(10) Pub. No.: US 2026/0125388 A1
(43) Pub. Date: May 7, 2026

(54) GABA(A) RECEPTOR MODULATORS AND METHODS TO CONTROL SMOOTH MUSCLE CONTRACTION AND INFLAMMATION

(71) Applicants: UWM Research Foundation, Inc., Milwaukee, WI (US); The Trustees of Columbia University in the City of New York, New York, NY (US)

(72) Inventors: Alexander E. Arnold, Milwaukee, WI (US); Michelle J. Meyer, Milwaukee, WI (US); Daniel A. Webb, Madison, WI (US); Douglas C. Stafford, Stoughton, WI (US); Charles W. Emala, Woodcliff Lake, NJ (US)

(21) Appl. No.: 19/117,919

(22) PCT Filed: Oct. 4, 2023
(86) PCT No.: PCT/US2023/075942

§ 371 Date: Apr. 2, 2025

Related U.S. Application Data
(60) Provisional application No. 63/413,044, filed on Oct. 4, 2022.

(51) Int. Cl. C07D 487/10; A61K 31/5517; A61P 3/10; A61P 11/00; A61P 11/06; A61P 13/10; A61P 17/00; A61P 19/02; C07D 487/04

(52) U.S. Cl. CPC C07D 487/10; A61K 31/5517; A61P 3/10; A61P 11/00; A61P 11/06; A61P 13/10; A61P 17/00; A61P 19/02; C07D 487/04

(57) ABSTRACT

Substituted [benzo[f]imidazo[1,5-a][1,4]diazepine]-3-carboxylic acid compounds are GABA(A) receptor modulators and have utility to treat inflammation and/or inhibit smooth muscle contraction.

### RELATED APPLICATIONS

This application claims priority to U.S. Provisional Application No. 63/413,044, filed Oct. 4, 2022, which is hereby incorporated by reference in its entirety.

### TECHNICAL FIELD

The present disclosure relates to compounds, compositions, and methods for treating or preventing inflammatory disorders and disorders associated with smooth muscle contraction.

### BACKGROUND

Chronic inflammatory diseases are the most significant cause of death in the world and The World Health Organization (WHO) ranks chronic diseases as the greatest threat to human health. Inflammation can be acute, resulting from tissue damage due to trauma, microbial invasion, or noxious compounds. It starts rapidly, can become severe in a short time and symptoms may last for a few days for example cellulitis or acute pneumonia. Inflammation can be chronic with symptoms lasting for prolonged periods of weeks, years, or lifelong. Generally, the extent and effects of chronic inflammation vary with the inducing agent and the ability of the body to repair and overcome the damage. Inflammation involves a complex network of many mediators, a variety of cells, and can be manifested in multiple anatomical sites. Treatment of the chronic inflammation includes steroidal and non-steroidal anti-inflammatory agents, and biologic therapeutics. The chronic use of these drugs is reported to cause severe adverse effects like gastrointestinal, cardiovascular, central nervous system, and renal abnormalities, among others. Inflammation can also contribute to other disease conditions such as abnormal smooth muscle contraction, such as airway hyperresponsiveness associated with lung inflammation, or itch associated with atopic dermatitis.

### SUMMARY

In one aspect, the invention provides a compound of formula (I), or a pharmaceutically acceptable salt thereof,

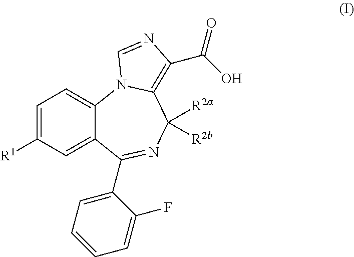

wherein
R¹ is H, halogen, CF₃, —OC₁₋₄alkyl, —C≡CH, or cyclopropyl; and R^{2a} and R^{2b} are independently C₁₋₄alkyl, CF₃, or CCl₃; or R^{2a} and R^{2b} together with the carbon to which each attaches form a 3- to 6-membered saturated carbocyclic ring, the carbocyclic ring being optionally substituted with 1-4 substituents independently selected from the group consisting of methyl and fluoro.

In another aspect, the present invention provides a pharmaceutical composition including a compound of formula (I) or (II), or a pharmaceutically acceptable salt thereof, and a pharmaceutically acceptable carrier. In some embodiments, the composition may be an oral, topical, injectable, or aerosol formulation.

In another aspect, the invention provides compounds of formula (II) and pharmaceutically acceptable salts thereof, for use in the therapeutic methods disclosed herein,

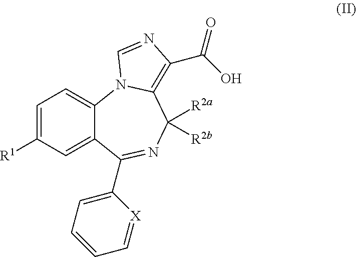

wherein
X is C—R^{x} or N;
R^{x} is halogen;
R¹ is H, halogen, CF₃, —OC₁₋₄alkyl, —C≡CH, or cyclopropyl; and
R^{2a} and R^{2b} are independently H, C₁₋₄alkyl, CF₃, or CCl₃; or R^{2a} and R^{2b} together with the carbon to which each attaches form a 3- to 6-membered saturated carbocyclic ring, the carbocyclic ring being optionally substituted with 1-4 substituents independently selected from the group consisting of methyl and fluoro.

In another aspect, the invention provides a method of reducing airway constriction comprising administering an effective amount of a compound or composition of formula (I) or (II), or a pharmaceutically acceptable salt thereof to a subject in need thereof. In some embodiments, compounds of formula (I) or (II) have reduced benzodiazepine-type CNS effects in a subject compared to diazepam at therapeutic doses.

In another aspect, the invention provides a method of reducing inflammation comprising administering an effective amount of a compound or composition of formula (I) or (II), or a pharmaceutically acceptable salt thereof to a subject in need thereof. In some embodiments, compounds of formula (I) or (II) have reduced benzodiazepine-type CNS effects in a subject compared to diazepam at therapeutic doses.

In another aspect, the invention provides a method of reducing itch, comprising administering an effective amount of a compound or composition of formula (I) or (II), or a pharmaceutically acceptable salt thereof to a subject in need thereof. In some embodiments, compounds of formula (I) or (II) have reduced benzodiazepine-type CNS effects in a subject compared to diazepam at therapeutic doses.

In another aspect, the invention provides a method of reducing development of disease in a subject having risk factors associated with inflammation comprising administering an effective amount of a compound or composition of formula (I) or (II), or a pharmaceutically acceptable salt thereof to a subject in need thereof. In some embodiments, compounds of formula (I) or (II) have reduced benzodiazepine-type CNS effects in a subject compared to diazepam at therapeutic doses.

In another aspect, the invention provides a method of treating inflammation comprising administering an effective amount of a compound or composition of formula (I) or (II), or a pharmaceutically acceptable salt thereof to a subject in need thereof. In some embodiments, compounds of formula (I) or (II) have reduced benzodiazepine-type CNS effects in a subject compared to diazepam at therapeutic doses.

In some aspects, the compounds of the present invention selectively target the a₄ or as and as subunits of GABA_{A}Rs. In some aspects, the compounds of the present invention are allosteric modulators of the GABA_{A}Rs that are selective for the a₄ or a₅ and as benzodiazepine allosteric modulatory sites on GABA_{A}Rs. In some aspects, the compounds of the present invention may have limited inability to cross the blood-brain barrier.

### BRIEF DESCRIPTION OF THE FIGURES

FIG. 1 shows the effect of compounds on sensorimotor coordination. Swiss Webster mice were tested on a rotarod at 15 rpm for 3 minutes at 10, 30, and 60 min following compound exposure.

FIG. 2. shows the effect of compounds on airway smooth muscle contractile force in guinea pig tracheal rings.

FIG. 3 shows the effect of compounds on specific airway resistance in mice.

FIG. 4 shows the effect of oral administration of PI-301 to reduce the increase of ear thickness due to MC903 in a mouse atopic dermatitis model.

FIG. 5 shows the effect of oral administration of PI-301 to reduce the number of scratches in a mouse atopic dermatitis model.

FIG. 6 shows the thicknesses of the dermis and epidermis in mice treated with PI-301 compared to vehicle (veh) in a mouse atopic dermatitis model.

FIG. 7 shows the effect of PI-301 on IL-6, IL-10, IL-13 and thymic stromal lymphopoietin (TSLP) induction in mice challenged with MC903 and compared to vehicle treatment.

FIG. 8 shows the acute treatment effects of oclacitinib and PI-301 in a MC903 mouse model on the number of scratches.

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemisty, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl). Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “halogen” or “halo,” as used herein, means Cl, Br, I, or F.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

### 2. COMPOUNDS

Compounds useful in the invention are set forth in the following numbered embodiments. The first embodiment is denoted E1, another embodiment is denoted E2 and so forth.
E1. A compound of formula (I), or a pharmaceutically acceptable salt thereof,

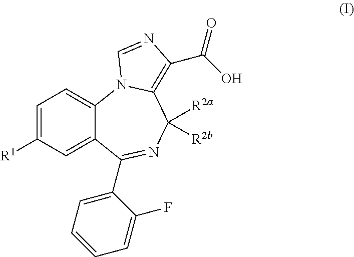

wherein
R¹ is H, halogen, CF₃, —OC₁₋₄alkyl, —C≡CH, or cyclopropyl; and
R^{2a} and R^{2b} are independently C₁₋₄alkyl, CF₃, or CCl₃; or R² and R^{2b} together with the carbon to which each attaches form a 3- to 6-membered saturated carbocyclic ring, the carbocyclic ring being optionally substituted with 1-4 substituents independently selected from the group consisting of methyl and fluoro.
E2. The compound of E1, or a pharmaceutically acceptable salt thereof, wherein R^{2a} and R^{2b} are independently C₁₋₄alkyl, CF₃, or CCl₃.
E3. The compound of E2, or a pharmaceutically acceptable salt thereof, wherein R^{2a} and R^{2b} are independently C₁₋₄alkyl.
E4. The compound of E3, or a pharmaceutically acceptable salt thereof, wherein R^{2a} and R^{2b} are methyl.
E5. The compound of E1, or a pharmaceutically acceptable salt thereof, wherein R^{2a} and R^{2b} together with the carbon to which each attaches form the optionally substituted 3- to 6-membered saturated carbocyclic ring.
E6. The compound of E1 or E5, or a pharmaceutically acceptable salt thereof, wherein R^{2a} and R^{2b} together with the carbon to which each attaches form a cyclopropyl.
E7. The compound of E1 or E5, or a pharmaceutically acceptable salt thereof, wherein R^{2a} and R^{2b} together with the carbon to which each attaches form a cyclobutyl.
E8. The compound of E1 or E5, or a pharmaceutically acceptable salt thereof, wherein R^{2a} and R^{2b} together with the carbon to which each attaches form a cyclopentyl.
E9. The compound of E1 or E5, or a pharmaceutically acceptable salt thereof, wherein R^{2a} and R^{2b} together with the carbon to which each attaches form a cyclohexyl.
E10. The compound of any of E1-E9, or a pharmaceutically acceptable salt thereof, wherein R¹ is H.
E11. The compound of any of E1-E9, or a pharmaceutically acceptable salt thereof, wherein R¹ is Br.
E12. The compound of any of E1-E9, or a pharmaceutically acceptable salt thereof, wherein R¹ is —C≡CH.
E13. The compound of any of E1-E9, or a pharmaceutically acceptable salt thereof, wherein R¹ is cyclopropyl.
E14. The compound of E1, or a pharmaceutically acceptable salt thereof, wherein the compound is selected from the group consisting of:

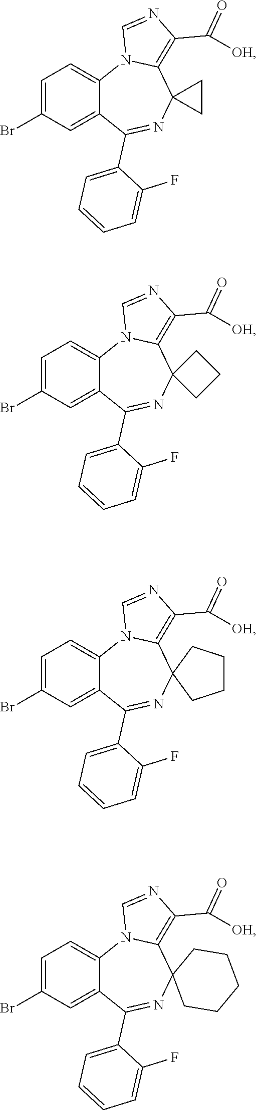 [SMILES: O=C(O)c1ncn2c1C1(CC1)N=C(c1ccccc1F)c1cc(Br)ccc1-2 ; O=C(O)c1ncn2c1C1(CCC1)N=C(c1ccccc1F)c1cc(Br)ccc1-2 ; O=C(O)c1ncn2c1C1(CCCC1)N=C(c1ccccc1F)c1cc(Br)ccc1-2 ; O=C(O)c1ncn2c1C1(CCCCC1)N=C(c1ccccc1F)c1cc(Br)ccc1-2]

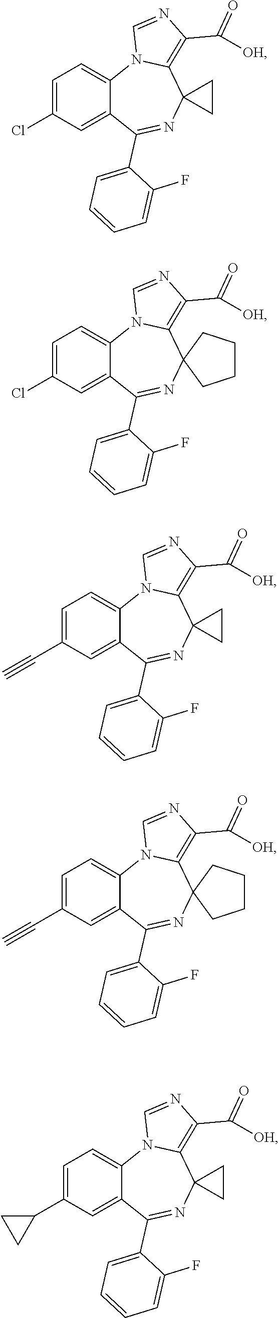 [SMILES: O=C(O)c1ncn2c1C1(CC1)N=C(c1ccccc1F)c1cc(Cl)ccc1-2 ; O=C(O)c1ncn2c1C1(CCCC1)N=C(c1ccccc1F)c1cc(Cl)ccc1-2 ; C#Cc1ccc2c(c1)C(c1ccccc1F)=NC1(CC1)c1c(C(=O)O)ncn1-2 ; C#Cc1ccc2c(c1)C(c1ccccc1F)=NC1(CCCC1)c1c(C(=O)O)ncn1-2 ; O=C(O)c1ncn2c1C1(CC1)N=C(c1ccccc1F)c1cc(C3CC3)ccc1-2]

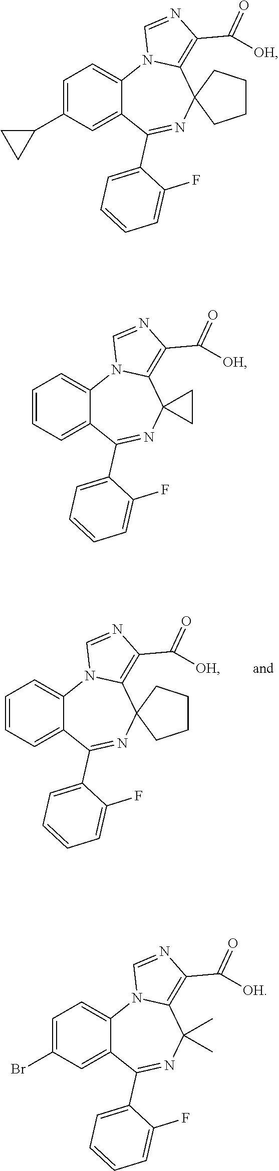 [SMILES: O=C(O)c1ncn2c1C1(CCCC1)N=C(c1ccccc1F)c1cc(C3CC3)ccc1-2 ; O=C(O)c1ncn2c1C1(CC1)N=C(c1ccccc1F)c1ccccc1-2 ; O=C(O)c1ncn2c1C1(CCCC1)N=C(c1ccccc1F)c1ccccc1-2 ; CC1(C)N=C(c2ccccc2F)c2cc(Br)ccc2-n2cnc(C(=O)O)c21]

E15. A pharmaceutical composition comprising a compound of any of E1-E14, or a pharmaceutically acceptable salt thereof, and a pharmaceutically acceptable carrier.

Compound names can be assigned by using Struct=Name naming algorithm as part of CHEMDRAW® ULTRA.

The compound may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this invention. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods.

It should be understood that the compound may possess tautomeric forms, as well as geometric isomers, and that these also constitute an aspect of the invention.

In the compounds of formula (I) and (II), and any subformulas, any “hydrogen” or “H,” whether explicitly recited or implicit in the structure, encompasses hydrogen isotopes ¹H (protium) and ²H (deuterium).

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, thrichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

Examples of a basic addition salt, the neutral form, a zwitterionic form, and acid addition salts are depicted. The acid addition salt may exist in closed and/or open forms, the latter forming in the presence of water to add a molecule of H₂O. See Roni et al., Mol. Pharmaceutics 2020, 17, 1182-1192.

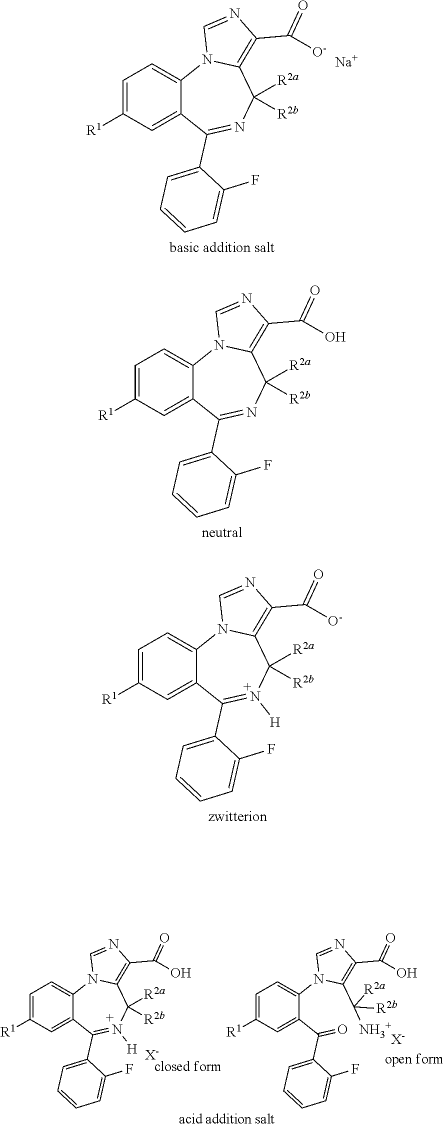

Compounds of formulae (I) and (11) may be synthesized using commercially available starting materials. Exemplary syntheses are summarized in Schemes 1-4 and in Clayton, T., et al., A review of the updated pharmacophore for the alpha 5 GABAA benzodiazepine receptor model. Int J Med Chem. 2015:430248. 2015.

Compounds of formula (I) and (II) may be prepared by synthetic processes or by metabolic processes. Preparation of the compounds by metabolic processes includes those occurring in the human or animal body (in vivo) or processes occurring in vitro.

### Abbreviations

AcOH acetic acid
t-BuOK potassium tert-butoxide
ClPO(OEt)₂ diethyl chlorophosphate
CNCH₂CO₂Et ethyl isocyanoacetate
eq equivalent(s)
EtOAc ethyl acetate
Et₃N or TEA triethylamine
h or hr hour(s)
IPA isopropyl alcohol
MeOH methyl alcohol
min minute(s)
Pd(OAc)₂ palladium acetate
P(o-tolyl)₃ tris(2-methylphenyl)phosphane
rt room temperature
TBAF tetrabutylammonium fluoride
TFA trifluoroacetic acid
THE tetrahydrofuran
TIPS triisopropylsilyl
TLC thin layer chromatography

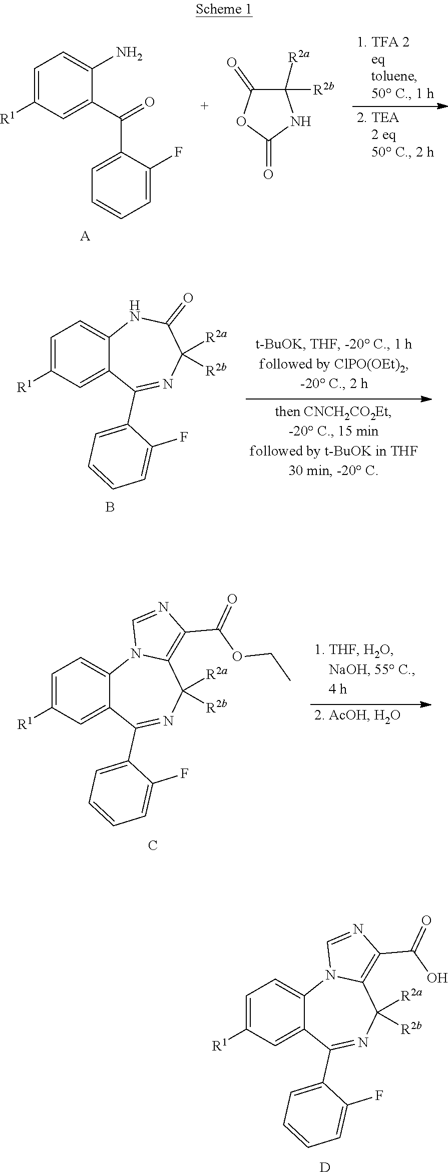

Scheme 1 shows processes that may be used for converting starting amino-benzophenones A to compounds D.

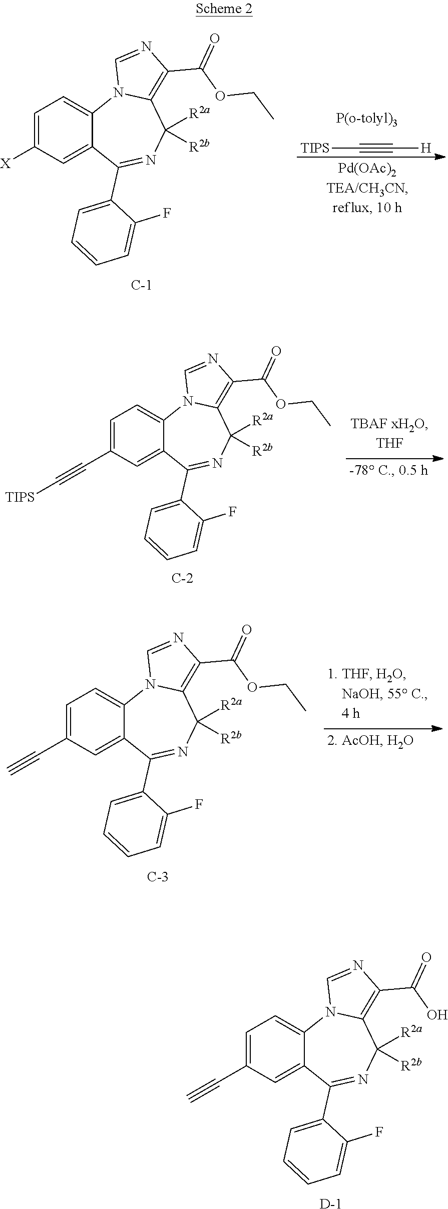

Scheme 2 shows processes that may be used for converting compounds C-1, wherein X is chloro or bromo, to compounds D-1.

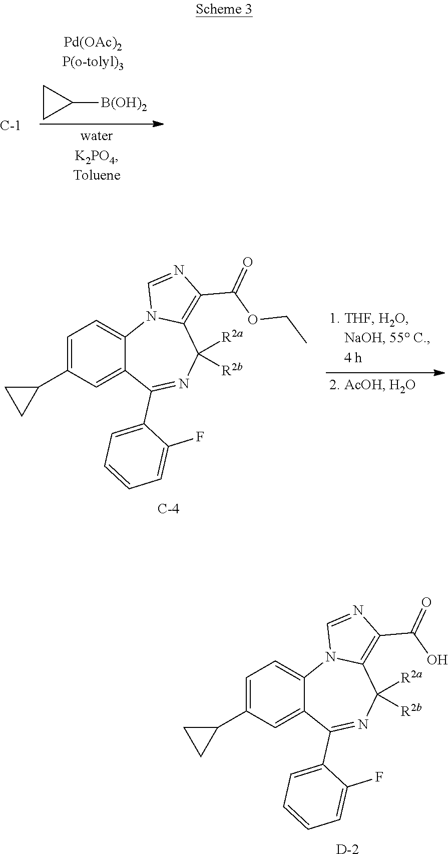

Scheme 3 shows processes that may be used for converting compounds C-1, wherein X is chloro or bromo, to compounds D-2.

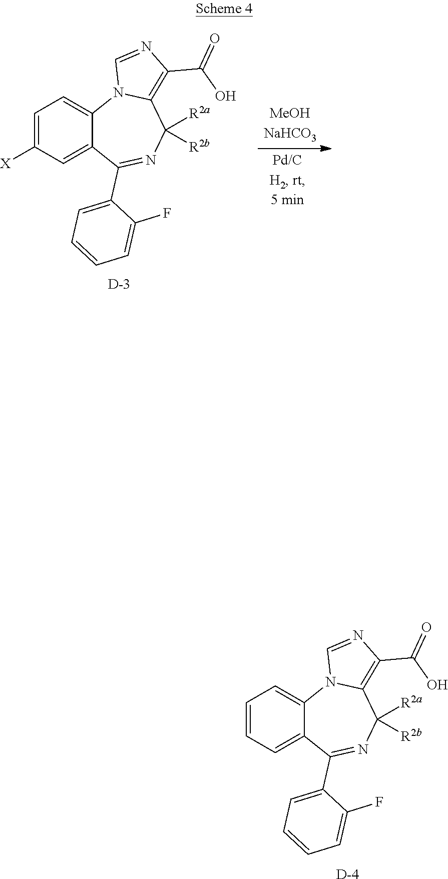

Scheme 4 shows a process that may be used for converting compounds D3, wherein X is chloro or bromo, to compounds D-4.

The compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry”, 5th edition (1989), by Furniss, Hannaford, Smith, and Tatchell, pub. Longman Scientific & Technical, Essex CM20 2JE, England.

A disclosed compound may have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Optimum reaction conditions and reaction times for each individual step can vary depending on the particular reactants employed and substituents present in the reactants used. Specific procedures are provided in the Examples section. Reactions can be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4^{th} ed.), John Wiley & Sons, NY (2006), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

### 3. PHARMACEUTICAL COMPOSITIONS

The disclosed compounds may be incorporated into pharmaceutical compositions suitable for administration to a subject (such as a patient, which may be a human or non-human animal, such as a mammal).

The pharmaceutical compositions may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention (e.g., a compound of formula (I)) are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

It will be appreciated that appropriate dosages of the compounds, and compositions comprising the compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician. In general, a suitable dose of the compound is in the range of about 100 μg to about 250 mg per kilogram body weight of the subject per day.

The composition may be administered once, on a continuous basis (e.g. by an intravenous drip), or on a periodic/intermittent basis, including about once per hour, about once per two hours, about once per four hours, about once per eight hours, about once per twelve hours, about once per day, about once per two days, about once per three days, about twice per week, about once per week, and about once per month. The composition may be administered until a desired reduction of symptoms is achieved.

The present compounds, compositions, and methods may be administered as part of a therapeutic regimen along with other treatments appropriate for the particular injury or disease being treated.

For example, a therapeutically effective amount of a compound of formula (I) and (II), may be about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds and their physiologically acceptable salts and solvates may be formulated for administration by, for example, solid dosing, eyedrop, in a topical oil-based formulation, injection, inhalation (either through the mouth or the nose), implants, or oral, buccal, parenteral, or rectal administration. Techniques and formulations may generally be found in “Remington's Pharmaceutical Sciences”, (Meade Publishing Co., Easton, Pa.). Therapeutic compositions must typically be sterile and stable under the conditions of manufacture and storage.

The route by which the disclosed compounds are administered, and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmelose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%.

Suitable suspending agents include AVICEL RC-591 (from FMC Corporation of Philadelphia, PA) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of active [e.g., compound of formula (I)] and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose and cellulose. Specific binders include starch, gelatin, and sucrose. Specific disintegrants include alginic acid and croscarmelose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound [e.g., a compound of formula (I)], and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this invention.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT coatings (available from Rohm & Haas G.M.B.H. of Darmstadt, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

For parenteral administration, the agent can be dissolved or suspended in a physiologically acceptable diluent, such as, e.g., water, buffer, oils with or without solubilizers, surface-active agents, dispersants or emulsifiers. As oils for example and without limitation, olive oil, peanut oil, cottonseed oil, soybean oil, castor oil and sesame oil may be used. More generally spoken, for parenteral administration, the agent can be in the form of an aqueous, lipid, oily or other kind of solution or suspension or even administered in the form of liposomes or nano-suspensions.

The term “parenterally,” as used herein, refers to modes of administration which include intravenous, intramuscular, intraperitoneal, intrasternal, subcutaneous and intraarticular injection and infusion.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The pharmaceutical compositions of the present invention may also be administered by nasal aerosol or inhalation through the use of a nebulizer, a dry powder inhaler or a metered dose inhaler. Such compositions are prepared according to techniques well-known in the art of pharmaceutical formulation and may be prepared as solutions in saline, employing benzyl alcohol or other suitable preservatives, absorption promoters to enhance bioavailability, hydrofluorocarbons, and/or other conventional solubilizing or dispersing agents.

Aerosol propellants are required where the pharmaceutical composition is to be delivered as an aerosol under significant pressure. Such propellants include, e.g., acceptable fluorochlorohydrocarbons such as dichlorodifluoromethane, dichlorotetrafluoroethane, and trichloromonofluoromethane; nitrogen; or a volatile hydrocarbon such as butane, propane, isobutane or mixtures thereof.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound (e.g., a compound of formula (I)), and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the medicament. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified Montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% to about 0.5%, particularly, about 0.001% to about 0.1%.

Suitable pH adjusting additives include HCl or NaOH in amounts sufficient to adjust the pH of a topical pharmaceutical composition.

### 4. METHODS OF TREATMENT

### A. Diseases and Disorders

Compounds of the invention may be useful in the treatment of various inflammatory conditions and/or airway constriction, as listed below.

Acute inflammatory diseases: Septic shock, sepsis, systemic inflammatory response syndrome (SIRS), hemorrhagic shock, shock states induced by cytokine therapy (interleukin-2, tumor necrosis factor, immune checkpoint inhibition), organ transplantation and transplant rejection, head trauma, acute lung injury, acute respiratory distress syndrome (ARDS), inflammatory skin conditions such as sunburn, inflammatory eye conditions such as uveitis, glaucoma and conjunctivitis.

Chronic inflammatory diseases, in particular chronic inflammatory diseases of peripheral organs and the CNS: gastrointestinal inflammatory diseases such as Crohn's disease, inflammatory bowel disease, and ulcerative colitis; lung inflammatory diseases such as asthma, chronic bronchitis, emphysema and COPD; inflammatory diseases of the upper respiratory tract such as allergic rhinitis and allergic sinusitis; inflammatory eye conditions such as allergic conjunctivitis; arthritic disorders such as rheumatoid arthritis, osteoarthritis and gouty arthritis; heart disorders such as cardiomyopathy and myocarditis; atherosclerosis; neurogenic inflammation; skin diseases such as psoriasis, pruritus, dermatitis and eczema; diabetes; glomerulonephritis; Parkinson's disease; amyotrophic lateral sclerosis (ALS), multiple sclerosis; necrotizing vasculitides such as polyarteritis nodosa; serum sickness; Wegener's granulomatosis; Kawasaki's syndrome; headaches such as migraine, chronic tension headaches, cluster, and vascular headaches; urological disorders such as overactive bladder and cystitis; and myocardial and cerebral ischemia/reperfusion injury.

A respiratory disease or condition including: asthmatic conditions including allergen-induced asthma, exercise-induced asthma, pollution-induced asthma, cold-induced asthma, and viral-induced-asthma; chronic obstructive pulmonary diseases including chronic bronchitis with normal airflow, chronic bronchitis with airway obstruction (chronic obstructive bronchitis), emphysema, asthmatic bronchitis, and bullous disease; and other pulmonary diseases involving inflammation including bronchioectasis cystic fibrosis, pigeon fancier's disease, farmer's lung, acute respiratory distress syndrome, pneumonia, aspiration or inhalation injury, fat embolism in the lung, acidosis inflammation of the lung, acute pulmonary edema, acute mountain sickness, acute pulmonary hypertension, persistent pulmonary hypertension of the newborn, perinatal aspiration syndrome, hyaline membrane disease, acute pulmonary thromboembolism, heparin-protamine reactions, sepsis, status asthmaticus and hypoxia.

Inflammatory pain, pain syndromes such as ocular pain, pain such as surgical analgesia, or as an antipyretic for the treatment of fever, post-surgical pain for various surgical procedures including post-cardiac surgery, dental pain/dental extraction, pain resulting from cancer, muscular pain, mastalgia, pain resulting from dermal injuries, lower back pain, headaches of various etiologies, including migraine, and the like, pain-related disorders such as tactile allodynia and hyperalgesia.

Insulin resistance and other metabolic disorders such as atherosclerosis that are typically associated with an exaggerated inflammatory signaling.

Arthritis, including but not limited to rheumatoid arthritis, spondyloarthropathies, gouty arthritis, osteoarthritis, systemic lupus erythematosus, juvenile arthritis, acute rheumatic arthritis, enteropathic arthritis, neuropathic arthritis, psoriatic arthritis, and pyogenic arthritis.

Osteoporosis and other related bone disorders.

Gastrointestinal conditions such as reflux esophagitis, diarrhea, inflammatory bowel disease, Crohn's disease, gastritis, irritable bowel syndrome, ulcerative colitis, celiac disease, gastritis, pancreatitis, proctitis, hepatitis, diverticulitis, and tropical sprue.

Pulmonary inflammation, such as that associated with viral infections and cystic fibrosis.

Compounds of the instant invention may be useful to prevent or treat a plurality of dermatologic diseases or conditions, including but not limited to, dermatitis, atopic dermatitis, rash, pruritis, ankylosing spondylitis, eczema, acne, dandruff, cellulitis, psoriasis, rosacea, hives, shingles, lupus erythematosus, lichen planus, dermatitides, vasculitis, bullous diseases, and vitiligo.

Atopic dermatitis (AD) is a multifaceted, chronic relapsing inflammatory skin disease that is commonly associated with other atopic manifestations such as food allergy, allergic rhinitis, and asthma (N Engl J Med, 2008; 358(14):1483-1494; J Invest Dermatol, 2017; 137(1):26-30). Incidence of AD, also referred to as atopic eczema (the word “eczema” is a broad term that refers to various conditions causing inflammation of the skin), has increased 2- to 3-fold in industrialized nations since the 1970s, with approximately 15% to 20% of children and 1% to 3% of adults affected worldwide (Ann Nutr Metab. 2015; 66(suppl 1):8-16; J Am Acad Dermatol, 2014; 70(2):338-351). Population-based studies in the United States suggest that prevalence is about 10.7% for children and 7.2% for adults. Onset of disease commonly presents by 5 years of age, with the highest incidence occurring between the ages of 3 and 6 months, but it can occur at any age. Approximately 60% of patients develop disease in the first year of life and 90% within the first 5 years of life (J Am Acad Dermatol, 2014; 70(2):338-351). Twenty percent of children who develop AD before 2 years of age will have persisting symptoms of disease; 17% will have intermittent symptoms by 7 years of age. It is clear that AD is a chronic disease that is burdensome for many patients. A 2007 study further supports this claim, as an estimated 17.8 million persons, mostly undiagnosed, are living with AD in the United States (Dermatitis, 2007; 18(2):82-91).

AD disease burden often impacts overall quality of life and the social, academic, and occupational realms. The burden of AD is not limited to just the patient, because AD is a chronic relapsing skin disease that can persist into adulthood and burden of disease is frequently experienced by the patient's family. Direct costs include, but are not limited to, prescription and nonprescription costs, healthcare provider visits, hospital and emergency department visits, and hospitalizations. Indirect costs associated with AD include absenteeism from work, school, and physical activities; decreased productivity (presenteeism); and decreased quality of life (primarily due to sleep disturbance from itching, absenteeism, and time related to care) (Pediatr Dermatol, 2005; 22(3):192-199). A 2010 National Health Interview Study conducted in the United States estimated that 75% of people with eczema had visited a doctor at least once in the last year (J Invest Dermatol, 2015; 135(1):56-66).

AD is considered to be a chronic relapsing inflammatory skin condition. As a result, it generally presents in 3 different clinical phases: 1) acute AD (a vesicular, weeping, crusting eruption); 2) subacute AD (dry, scaly, erythematous papules and plaques); and 3) chronic AD (lichenification, thickening, from repeated scratching). Itch is the major symptom associated with impact on quality of life. Itch has been associated with mental distress and increased risk for suicidal ideation in those with AD (J Invest Dermatol, 2014; 134(7):1847-1854). Sleep disturbance is a frequent consequence of itch and is experienced by approximately two-thirds of patients with AD. Children with AD who experience sleep disturbances are associated with higher rates of developing attention-deficit/hyperactivity disorder, headaches, and short stature. Sleep disturbances experienced by adults with AD are associated with poor overall health perception (J Invest Dermatol, 2015; 135(1):56-66). Patients with atopic dermatitis are also more likely to develop ear infections (27% vs. 22%), streptococcal pharyngitis (8% vs. 3%), and urinary tract infections (8% vs. 3%).

Two major theories have been proposed to explain the cause of AD, the inside-out and outside-in hypotheses (Cutis, 2015; 96(6):359-361). The inside-out hypothesis proposes that allergic triggering leads to a weakened skin barrier that furthers allergen introduction and presentation. This would suggest that inflammation is the culprit for an impaired skin barrier, leading to increased penetration of allergens and microbes causing a reaction. The outside-in hypothesis proposes that the impaired skin barrier precedes AD and is required for immune dysregulation to occur. For example, the down-regulation of filaggrin (FLG), required for proper skin barrier function, could make the skin more susceptible to immune dysregulation and lead to AD. It is unlikely that the two theories are exclusive, and both most likely play a role in the pathogenesis of AD.

Certain genes associated with the immune system, for example cytokines that regulate immunoglobulin E (IgE) synthesis such as interleukin-4 (IL-4), interleukin-5 (IL-5), interleukin-12 (IL-12), interleukin-13 (IL-13), and granulocyte-macrophage colony-stimulating factor, have been associated with AD (N Engl J Med, 2008; 358(14). Cytokines are mainly produced by type 1 and type 2 T helper lymphocytes (TH1 and TH2, respectively). TH1 cytokines (IL-12 and interferon-suppress IgE production, and TH2 cytokines (IL-5 and IL-13) increase IgE production. Patients with AD have a genetically determined dominance of TH2 cells that may decrease expression of FLG and other molecules found in the skin barrier.

The “atopic march” describes the tendency for AD to precede the development of other atopic diseases such as food allergies, asthma, and allergic rhinitis in a temporal sequence (Immunol Allergy Clin North Am, 2005; 25(2):231-246). Patients with atopic dermatitis, allergic rhinitis, and allergic asthma are considered to have the atopic triad. Approximately one-third of patients with AD develop asthma, and two-thirds develop allergic rhinitis (J Allergy Clin Immunol, 2007; 120(3):565-569; Immunol Allergy Clin North Am, 2010; 30(3):269-280).

AD may be triggered by viral infections, food allergens, cosmetics, fragrance, weather, and other causes. Extremes of hot and cold weather are poorly tolerated by patients with AD and can lead to sweating and dry skin, respectively, initiating pruritus. Exposure to environmental allergens such as dust mites, pollens, molds, cigarette smoke, and dander from animals may exacerbate symptoms of AD (Cutis, 2016; 97(5):326-329). Food allergens may contribute to AD and the most commonly allergenic foods are eggs, milk, peanuts, wheat, soy, tree nuts, shellfish, and fish.

The goal of treatment in AD is to reduce symptoms, prevent exacerbations, treat superinfection, minimize treatment risks, and restore the integrity of the skin. In patients with mild disease, treatment goals may be achieved with topical therapies alone, unlike in patients with moderate to severe disease, whose management is challenging. Long-standing topical therapies are not sufficient for treating severe AD or to improve quality of life. Systemic immunomodulating agents are limited by side effects and by their effectiveness and biologics are costly and most applicable for disease that is resistant to other treatment modalities.

The American Academy of Dermatology has created simple diagnostic criteria based on symptoms and physical examination findings. Maintenance therapy consists of liberal use of emollients and daily bathing with soap-free cleansers. Use of topical corticosteroids is the first-line treatment for AD flare-ups. Pimecrolimus and tacrolimus are topical calcineurin inhibitors that can be used in conjunction with topical corticosteroids as first-line treatment. Ultraviolet phototherapy is a safe and effective treatment for moderate to severe AD when first-line treatments are not adequate. Anti-staphylococcal antibiotics are effective in treating secondary skin infections. Oral antihistamines are not recommended because they do not reduce pruritus. Evidence is lacking to support the use of integrative medicine in the treatment of atopic dermatitis. Newer medications approved by the U.S. Food and Drug Administration, such as crisaborole and dupilumab, can be effective in treating AD but are currently cost prohibitive for most patients.

Non-pharmacologic interventions such as the role of moisturizers and bathing practices help with treatment, maintenance, and prevention of flares. The application of moisturizers should be an integral part of the treatment of patients with AD. They are also components of maintenance therapy and prevention of flares. Bathing is suggested in patients with AD as part of treatment and maintenance; however, there is no standard for the frequency or duration of bathing appropriate for those with AD. Moisturizers should be applied soon after bathing to improve skin hydration. Limited use of non-soap cleansers (that are neutral to low pH, hypoallergenic, and fragrance-free) is recommended, and there are no data to support the use of bath water additives (oils, emollients, etc.). Wet-wrap therapy with or without topical corticosteroid can be recommended for patients with moderate to severe AD to decrease severity and water loss during flares. Topical corticosteroids (TCS) are used on both adults and pediatrics for the management of AD. A variety of factors must be considered to choose the proper TCS for treatment, such as patient preference and age. Based on patient risk factors and response, some monitoring may be required. Topical calcineurin inhibitors (TCI) can be used for the treatment of acute and chronic AD as well as maintenance therapy in both adults and children. They can serve as a steroid-sparing treatment; however, careful considerations must be made before prescribing. Patient education regarding TCI for AD is crucial since there are some adverse effects they may experience. Concomitant use of TCS and TCI can be used to treat AD. Topical antimicrobials and antiseptics such as bleach baths with intranasal mupirocin have been shown to be beneficial for AD patients; however, they are not routinely recommended. This therapy is mostly for patients with clinical signs of secondary bacterial infection to help reduce disease severity. Other forms of antimicrobial and antiseptic therapies have not been shown to be clinically helpful for AD. Topical antihistamines are not suggested for the treatment of AD due to the risk of absorption and contact dermatitis.

Phototherapy is typically used as a treatment for both acute and chronic AD in pediatric and adult patients. Phototherapy can be used as monotherapy or in combination with other topical therapies. However, caution must be taken due to drug interactions and increased risk of adverse effects. Phototherapy can be used in children; however, additional factors such as their psychological perspective may need to be considered when administering therapy.

Various factors must be considered when prescribing systemic agents, such as previous therapy failure or contraindications, as well as quality of life and disease severity. When using systemic agents, the minimal effective dose should be used, due to lack of optimal dosing, duration, or monitoring protocol guidance. Treatment is highly individualized and based on patient response, comorbidities, and history.

The following systemic therapies can be used off-label to treat AD. Some should only be considered as an alternative when other more commonly used off-label systemic therapies are not an option: cyclosporine, azathioprine, methotrexate, mycophenolate mofetil, and interferon gamma. Systemic steroids should be avoided, when possible, for the treatment of AD. They mainly serve for short-term bridge therapy to other systemic therapies or for acute severe exacerbations. There are limited data to support the use of oral antihistamines as a treatment of AD, they can be used to help with pruritus and some sedating antihistamines can help (short-term) with sleep loss due to AD.

Tissue damage in such diseases as vascular diseases, migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephritis, nephrotic syndrome, Behcet's syndrome, polymyositis, gingivitis, periodontis, hypersensitivity, swelling occurring after injury, ischemias including myocardial ischemia, cardiovascular ischemia, and ischemia secondary to cardiac arrest, and the like.

Central nervous system disorders such as central nervous system damage resulting from stroke, ischemias including cerebral ischemia (both focal ischemia, thrombotic stroke and global ischemia (for example, secondary to cardiac arrest), and trauma.

Systemic hypotension associated with septic and/or toxic hemorrhagic shock induced by a wide variety of agents; therapy with cytokines such as TNF, IL-1 and IL-2.

Cancers that express nitric oxide synthase, such as colorectal cancer, and cancer of the breast, lung, prostate, bladder, cervix and skin, neoplasias including but not limited to brain cancer, bone cancer, a leukemia, a lymphoma, epithelial cell-derived neoplasia (epithelial carcinoma) such as basal cell carcinoma, adenocarcinoma, gastrointestinal cancer such as lip cancer, mouth cancer, esophogeal cancer, small bowel cancer and stomach cancer, colon cancer, liver cancer, bladder cancer, pancreas cancer, ovary cancer, cervical cancer, lung cancer, breast cancer and skin cancer, such as squamous cell and basal cell cancers, prostate cancer, renal cell carcinoma, and other known cancers that effect epithelial cells throughout the body.

Ophthalmic diseases, such as glaucoma, retinal ganglion degeneration, ocular ischemia, retinitis, retinopathies, uveitis, ocular photophobia, and of inflammation and pain associated with acute injury to the eye tissue, glaucomatous retinopathy and/or diabetic retinopathy, post-operative inflammation or pain as from ophthalmic surgery such as cataract surgery and refractive surgery.

The invention further relates to a method of treating or preventing one of the above-mentioned diseases in a mammal, including a human, in need thereof comprising administering a therapeutically effective amount of at least one of the compounds according to the invention.

Especially, the invention relates to a method of treating or preventing a disease which is alleviated by inhibition of inducible nitric oxide synthase in a mammal, including a human, in need thereof comprising administering a therapeutically effective amount of at least one of the compounds according to the invention.

In particular, the invention relates to a method of treating or preventing an acute or chronic inflammatory disease, in particular sepsis, septic shock, systemic inflammatory response syndrome, hemorrhagic shock, shock states induced by cytokine therapy, asthma, chronic obstructive pulmonary disease, allergic rhinitis, cardiomyopathy or myocarditis, in a mammal, including a human, in need thereof comprising administering a therapeutically effective amount of at least one of the compounds according to the invention.

In the context of treating a disorder, the term “effective amount” as used herein refers to an amount of the compound or a composition comprising the compound which is effective, upon single or multiple dose administrations to a subject, in treating a cell, or curing, alleviating, relieving or improving a symptom of the disorder in a subject. An effective amount of the compound or composition may vary according to the application. In the context of treating a disorder, an effective amount may depend on factors such as the disease state, age, sex, and weight of the individual, and the ability of the compound to elicit a desired response in the individual. In an example, an effective amount of a compound is an amount that produces a pharmacologically useful change in a given parameter as compared to a control, such as in cells (e.g., a culture of cells) or a subject not treated with the compound.

One aspect of the invention provides a method of treating an inflammatory disease or disorder in a subject comprising administering to the subject, a therapeutically effective amount of a compound of any of formula (I) or (II), or a pharmaceutically acceptable salt thereof, as defined in any of embodiments E1-E40.

Another aspect of the invention provides a method of reducing smooth muscle contraction in a subject comprising administering to the subject, a therapeutically effective amount of a compound of any of formula (I) or (II), or a pharmaceutically acceptable salt thereof, as defined in any of embodiments E1-E40.

Another aspect of the invention provides use of a compound of any of formula (I) or (II), or a pharmaceutically acceptable salt thereof, as defined in any of embodiments E1-E40, in the manufacture of a medicament for treating an inflammatory disease or disorder.

Another aspect of the invention provides use of a compound of any of formula (I) or (II), or a pharmaceutically acceptable salt thereof, as defined in any of embodiments E1-E40, in the manufacture of a medicament for reducing smooth muscle contraction.

Another aspect of the invention provides a compound of any of formula (I) or (II), or a pharmaceutically acceptable salt thereof, as defined in any of embodiments E1-E40, for use in treating an inflammatory disease or disorder.

Another aspect of the invention provides a compound of any of formula (I) or (II), or a pharmaceutically acceptable salt thereof, as defined in any of embodiments E1-E40, for use in reducing smooth muscle contraction.

Therapeutic methods of the invention are set forth in the following numbered embodiments E16 through E40.
E16. A method of reducing inflammation in a subject suffering an inflammatory disease or disorder comprising administering to the subject, an amount of a compound of formula (II), or a pharmaceutically acceptable salt or composition thereof, that is effective to reduce inflammation in the subject,

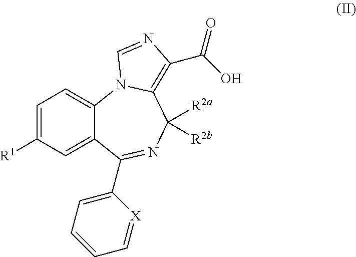

wherein
X is C—R^{x} or N;
R^{x} is halogen;
R¹ is H, halogen, CF₃, —OC₁₋₄alkyl, —C≡CH, or cyclopropyl; and
R^{2a} and R^{2b} are independently H, C₁₋₄alkyl, CF₃, or CCl₃; or R^{2a} and R^{2b} together with the carbon to which each attaches form a 3- to 6-membered saturated carbocyclic ring, the carbocyclic ring being optionally substituted with 1-4 substituents independently selected from the group consisting of methyl and fluoro.
E17. The method of E16, wherein the inflammatory disease or disorder is an acute inflammatory disease or disorder.
E18. The method of E16, wherein the inflammatory disease or disorder is a chronic inflammatory disease or disorder.
E19. The method of E16, wherein the inflammatory disease or disorder is a respiratory disease or condition.
E20. The method of E19, wherein the respiratory disease or condition is asthma, chronic obstructive pulmonary disease, bronchitis, emphysema, inflammatory diseases of the upper respiratory tract such as allergic rhinitis and allergic sinusitis, acute lung injury, acute respiratory distress syndrome (ARDS), lung infection, interstitial lung disease, or constrictive bronchiolitis.
E21. The method of E16, wherein the inflammatory disease or disorder is a digestive tract disease or condition.
E22. The method of E21, wherein the disease or disorder is an inflammatory bowel disease, Crohn's disease, ulcerative colitis, esophagitis, irritable bowel syndrome, celiac disease, gastritis, pancreatitis, proctitis, hepatitis, diverticulitis, or tropical sprue.
E23. The method of E16, wherein the inflammatory disease or disorder is a dermatologic disease or condition.
E24. The method of E23, wherein the disease or disorder is dermatitis, atopic dermatitis, rash, pruritis, eczema, acne, dandruff, cellulitis, psoriasis, rosacea, hives, shingles, lupus erythematosus, lichen planus, dermatitides, vasculitis, or bullous diseases.
E25. The method of E16, wherein the inflammatory disease or disorder is septic shock, sepsis, systemic inflammatory response syndrome (SIRS), hemorrhagic shock, shock states induced by cytokine therapy (interleukin-2, tumor necrosis factor, immune checkpoint inhibition), organ transplantation and transplant rejection, head trauma, or inflammatory eye conditions such as uveitis, glaucoma, and conjunctivitis.
E26. The method of E16, wherein the inflammatory disease or disorder is: an arthritic disorder such as rheumatoid arthritis, ankylosing spondylitis, osteoarthritis or gouty arthritis; a heart disorder such as cardiomyopathy or myocarditis; atherosclerosis; neurogenic inflammation; diabetes; glomerulonephritis; a urological disorder such as overactive bladder or cystitis; Parkinson's disease; Huntington's induced dementias; amyotrophic lateral sclerosis (ALS); multiple sclerosis; necrotizing vasculitides such as polyarteritis nodosa; serum sickness; Wegener's granulomatosis; Kawasaki's syndrome; headaches such as migraine, chronic tension headaches, cluster or vascular headaches; or myocardial and cerebral ischemia/reperfusion injury.
E27. A method of reducing airway constriction comprising administering to a subject in need thereof, an amount of a compound of formula (II), as defined in E16, or a pharmaceutically acceptable salt thereof, that is effective in reducing airway constriction in the subject.
E28. The method of any of E16-E27, wherein the compound, or pharmaceutically acceptable salt thereof is administered orally, topically, parenterally, or by inhalation.
E29. The method of any of E16-E28, wherein the subject is a human or non-human animal.
E30. The method of any of E16-E29, wherein:
X is C—R^{x} or N;
R^{x} is halogen;
R¹ is H, halogen, CF₃, —OC₁₋₄alkyl, —C≡CH, or cyclopropyl;
R^{2a} is C₁₋₄alkyl, CF₃, or CCl₃; and
R^{2b} is H, C₁₋₄alkyl, CF₃, or CCl₃.
E30.1. The method of E30, wherein R^{x} is fluoro.
E31. The method of E30 or E30.1, wherein R¹ is halogen, —C≡CH, or cyclopropyl.
E32. The method of E31, wherein R¹ is Cl, Br, or cyclopropyl.
E33. The method of any of E30-E32, wherein X is C—R^{x}.
E34. The method of any of E30-E32, wherein X is N.
E35. The method of any of E30-E34, wherein R^{2a} is C₁₋₄alkyl.
E35.1. The method of any of E16-E35, wherein the C₁₋₄alkyl at R^{2a} is methyl.
E35.2. The method of any of E16-E35, wherein the C₁₋₄alkyl at R^{2b} is ethyl.
E36. The method of any of E30-E35.2, wherein R^{2b} is H.
E37. The method of any of E30-E36, wherein the compound has (R) stereochemistry at R^{2a} and R^{2b}.
E38. The method of any of E30-E36, wherein the compound has (S) stereochemistry at R^{2a} and R^{2b}.
E39. The method of any of E30-E38, wherein the compound is selected from the group consisting of

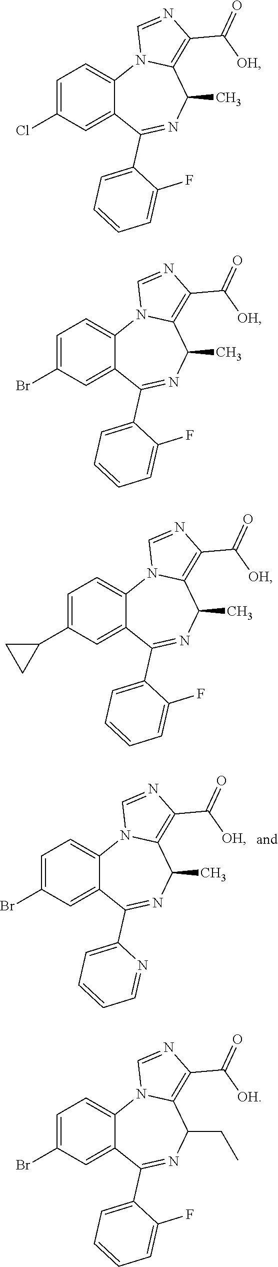 [SMILES: C[C@H]1N=C(c2ccccc2F)c2cc(Cl)ccc2-n2cnc(C(=O)O)c21 ; C[C@H]1N=C(c2ccccc2F)c2cc(Br)ccc2-n2cnc(C(=O)O)c21 ; C[C@H]1N=C(c2ccccc2F)c2cc(C3CC3)ccc2-n2cnc(C(=O)O)c21 ; C[C@H]1N=C(c2ccccn2)c2cc(Br)ccc2-n2cnc(C(=O)O)c21 ; CCC1N=C(c2ccccc2F)c2cc(Br)ccc2-n2cnc(C(=O)O)c21]

E39.1. The method of E39, wherein the compound is selected from the group consisting of

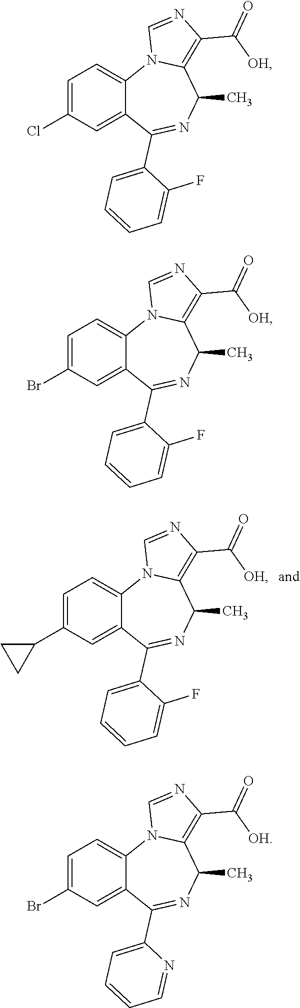 [SMILES: C[C@H]1N=C(c2ccccc2F)c2cc(Cl)ccc2-n2cnc(C(=O)O)c21 ; C[C@H]1N=C(c2ccccc2F)c2cc(Br)ccc2-n2cnc(C(=O)O)c21 ; C[C@H]1N=C(c2ccccc2F)c2cc(C3CC3)ccc2-n2cnc(C(=O)O)c21 ; C[C@H]1N=C(c2ccccn2)c2cc(Br)ccc2-n2cnc(C(=O)O)c21]

E39.2. The method of E39, wherein the compound is

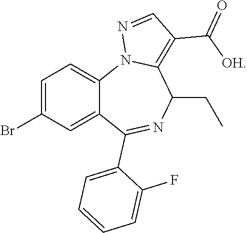 [SMILES: CCC1N=C(c2ccccc2F)c2cc(Br)ccc2-n2ncc(C(=O)O)c21]

E40. The method of any of E16-E29, wherein the compound or its pharmaceutically acceptable salt or composition is as defined in any of E1-E15.

### B. Combination Therapies

Additional therapeutic agent(s) may be administered simultaneously or sequentially with the disclosed compounds and compositions. Sequential administration includes administration before or after the disclosed compounds and compositions. In some embodiments, the additional therapeutic agent or agents may be administered in the same composition as the disclosed compounds. In other embodiments, there may be an interval of time between administration of the additional therapeutic agent and the disclosed compounds. In some embodiments, administration of an additional therapeutic agent with a disclosed compound may allow lower doses of the other therapeutic agents and/or administration at less frequent intervals. When used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of any one of formulas (I) or (II)

Specific, non-limiting examples of possible combination therapies include use of the compounds of the invention with: a) corticosteroids including betamethasone dipropionate (augmented and nonaugemented), betamethasone valerate, clobetasol propionate, diflorasone diacetate, halobetasol propionate, amcinonide, dexosimethasone, fluocinolone acetononide, fluocinonide, halocinonide, clocortalone pivalate, dexosimetasone, and flurandrenalide; b) non-steroidal anti-inflammatory drugs including diclofenac, ketoprofen, and piroxicam; c) muscle relaxants and combinations thereof with other agents, including cyclobenzaprine, baclofen, cyclobenzaprine/lidocaine, baclofen/cyclobenzaprine, and cyclobenzaprine/lidocaine/ketoprofen; d) anaesthetics and combinations thereof with other agents, including lidocaine, lidocaine/deoxy-D-glucose (an antiviral), prilocaine, and EMLA Cream [Eutectic Mixture of Local Anesthetics (lidocaine 2.5% and prilocaine 2.5%; an emulsion in which the oil phase is a eutectic mixture of lidocaine and prilocaine in a ratio of 1:1 by weight. This eutectic mixture has a melting point below room temperature and therefore both local anesthetics exist as a liquid oil rather than as crystals)]; e) expectorants and combinations thereof with other agents, including guaifenesin and guaifenesin/ketoprofen/cyclobenzaprine; f) antidepressants including tricyclic antidepressants (e.g., amitryptiline, doxepin, desipramine, imipramine, amoxapine, clomipramine, nortriptyline, and protriptyline), selective serotonin/norepinephrine reuptake inhibitors including (e.g, duloxetine and mirtazepine), and selective norepinephrine reuptake inhibitors (e.g., nisoxetine, maprotiline, and reboxetine), selective serotonin reuptake inhibitors (e.g., fluoxetine and fluvoxamine); g) anticonvulsants and combinations thereof, including gabapentin, carbamazepine, felbamate, lamotrigine, topiramate, tiagabine, oxcarbazepine, carbamezipine, zonisamide, mexiletine, gabapentin/clonidine, gabapentin/carbamazepine, and carbamazepine/cyclobenzaprine; h) antihypertensives including clonidine; i) opioids including loperamide, tramadol, morphine, fentanyl, oxycodone, levorphanol, and butorphanol; j) topical counter-irritants including menthol, oil of wintergreen, camphor, eucalyptus oil and turpentine oil; k) topical cannabinoids including selective and non-selective CB1/CB2 ligands; and other agents, such as capsaicin.

The present compounds may also be used in co-therapies, partially or completely, in place of other conventional anti-inflammatory therapies, such as together with steroids, NSAIDs, COX-2 selective inhibitors, 5-lipoxygenase inhibitors, LTB₄ antagonists and LTA₄ hydrolase inhibitors. The compounds of the subject invention may also be used to prevent tissue damage when therapeutically combined with antibacterial or antiviral agents.

The disclosed compounds may be included in kits comprising the compound [e.g., one or more compounds of formula (I)], a systemic or topical composition described above, or both; and information, instructions, or both that use of the kit will provide treatment for medical conditions in mammals (particularly humans). The information and instructions may be in the form of words, pictures, or both, and the like. In addition or in the alternative, the kit may include the medicament, a composition, or both; and information, instructions, or both, regarding methods of application of medicament, or of composition, preferably with the benefit of treating or preventing medical conditions in mammals (e.g., humans).

The compounds and processes of the invention will be better understood by reference to the following examples, which are intended as an illustration of and not a limitation upon the scope of the invention.

### 5. EXAMPLES

### Example 1. General Procedure for Compounds 2-8

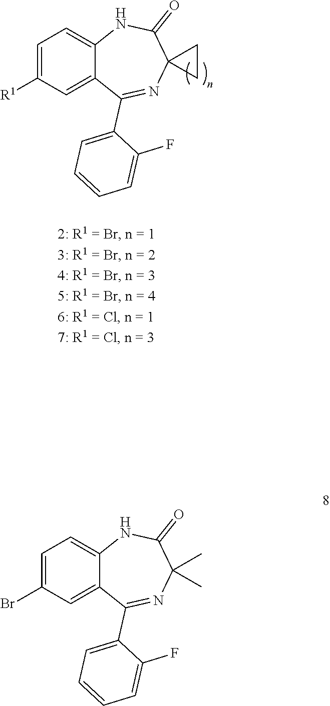

2-Amino-5-bromo-2′-fluorobenzophenone (3.0 g, 10.20 mmol) was added to anhydrous toluene (100 mL), followed by the addition of trifluoroacetic acid (1.56 mL, 20.40 mmol) dropwise over a period of 10 min, and the mixture was allowed to stir at room temperature for 30 min. 4,4-Dimethyloxazolidine-2,5-dione (1.98 g, 15.30 mmol) was added portion wise and the reaction was heated to 50° C. for 24 hr. After the majority of the starting material had been consumed by TLC (50% EtOAc:Hex), triethylamine (2.99 mL, 21.42 mmol) was added dropwise over a period of 15 min at which point fuming was observed in the reaction. The reaction was then heated to 100° C. for 24 hr at which point disappearance of the intermediate was observed via TLC (50% EtOAc:Hex). Upon cooling to room temperature, the solvent was removed under reduced pressure and the residue was dissolved in ethyl acetate (120 mL). The organic layer was washed with 5% aqueous sodium bicarbonate (120 mL), followed by 10% aqueous NaCl (120 mL). The organic layer was then dried with MgSO4 and the solvent was removed under reduced pressure. The residue was stripped with 10% EtOAc:Heptane (50 mL, 2×) followed by a trituration in 10% EtOAc:Heptane (80 mL) at 60° C. for 4 hr. The product 8 was collected by filtration to yield a light yellow solid (2.67 g, 72.3%): ¹H NMR (500 MHz, CDCl3) δ 9.51 (s, 1H), 7.47-4.45 (dd, J=3.62, 2.25 Hz, 1H), 7.44-7.41 (dt, J=3.34, 1.75 Hz, 1H), 7.38-7.33 (m, 1H), 7.19-7.18 (m, 1H), 7.17-7.14 (dt, J=3.23, 1.10 Hz, 1H), 7.01-6.97 (m, 1H), 6.96-6.94 (d, J=8.6 Hz, 1H), 1.41 (s, 6H); ¹³C NMR (126 MHz, CDCl3) δ 174.61 (s), 162.68 (s), 160.28 (d, 1JCF=250.70 Hz), 136.28 (s), 134.80 (s), 132.18 (d, 3JCF=1.13 Hz), 131.70 (d, 2JCF=8.24 Hz), 131.48 (d, 3JCF=2.55 Hz), 129.87 (d, 4JCF=0.89 Hz), 128.76 (d, 2JCF=13.22 Hz), 124.46 (d, 3JCF=3.55 Hz), 121.67 (s), 116.23 (d, 2JCF=21.49 Hz), 115.74 (s), 63.73 (s), 25.13 (s); ¹⁹F NMR (471 MHz, CDCl3) δ −113.18; HRMS (ESI/IT-TOF): m/z [M+H]+ calcd for C₁₇H₁₄BrFN₂O: 361.0346; found: 361.0312; HPLC Purity: 98.95%.

### Example 2. General Procedure for Compounds 9-15

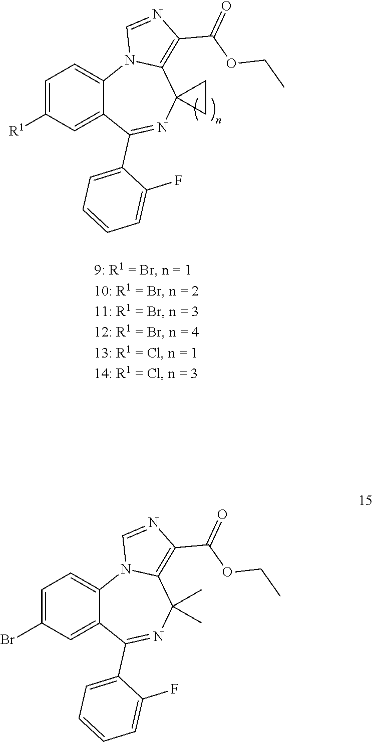

A three stopper RB flask was purged with nitrogen and vacuum 3×. Compound 8 (577.0 mg, 1.60 mmol) was dissolved in anhydrous tetrahydrofuran (6.8 mL) and added to the reaction flask. The mixture was cooled to −20° C. using a dry ice/IPA bath. A solution of 1M potassium tert-butoxide in anhydrous tetrahydrofuran (2.08 mL) was added dropwise over the course of 10 min, at which time the reaction color turned to a deep orange. Upon completion of the addition, the mixture was allowed to stir at −20° C. for 40 min. Diethyl chlorophosphate (0.32 mL, 2.24 mmol) was added dropwise over the course of 5 min while maintaining a temperature of −20° C. After 3.5 hr, no more conversion was observed via TLC (100% EtOAc) and ethyl isocyanoacetate (0.23 mL, 2.08 mmol) was added dropwise over the course of 5 min followed by the addition of a solution of 1M potassium tert-butoxide in anhydrous tetrahydrofuran (2.08 mL) at −20° C. The reaction was then warmed to room temperature for 2 hr at which point all of the intermediate had been consumed via TLC (100% EtOAc). The reaction was then quenched with 5% aqueous sodium bicarbonate (25 mL), and the product was extracted with ethyl acetate (25 mL). The organic layer was washed with 10% aqueous sodium bicarbonate (25 mL) followed by 20% aqueous NaCl (25 mL). The organic layer was then dried with MgSO4 and then concentrated under reduced pressure. The resulting residue was triturated with a 50% mixture of tert-butyl methyl ether in hexanes (12 mL) at 55° C. for 20 hr. The tert-butyl methyl ether/hexanes mixture was decanted, and the solid product was slurried in 100% hexanes (20 mL) at 55° C. for 4 hr. The desired product 15 was collected by filtration to yield an off-white solid (465.1 g, 63.8%): ¹H NMR (500 MHz, CDCl3) −25° C. δ 7.96 (s, 1H), 7.74-7.72 (dd, J=3.60, 2.25 Hz, 1H), 7.59-7.56 (m, 1H), 7.47-7.43 (m, 2H), 7.38-7.37 (d, J=2.15 Hz, 1H), 7.28-7.25 (dt, J=3.20, 0.95 Hz, 1H), 7.06-7.02 (m, 1H), 4.44-4.30 (m, 2H), 2.14 (s, 3H), 1.40-1.36 (t, J=7.15 Hz, 3H), 1.27 (s, 3H); ¹³C NMR (126 MHz, CDCl3) −25° C. δ 164.62 (s), 160.91 (s), 160.07 (d, 1JCF=250.72 Hz), 140.23 (s), 135.56 (s), 135.20 (s), 134.31 (s), 132.45 (s), 132.39 (s), 131.44 (d, 4JCF=1.61 Hz), 131.02 (s), 130.81 (s), 127.97 (d, 2JCF=12.48 Hz), 124.92 (d, 3JCF=3.13 Hz), 124.33 (s), 121.20 (s), 116.42 (d, 2JCF=21.13 Hz), 61.85 (s), 57.08 (s), 32.78 (s), 23.13 (s), 14.38 (d, J=1.27 Hz); ¹⁹F NMR (471 MHz, CDCl3) −25° C. δ −112.13; HRMS (ESI/IT-TOF): m/z [M+H]+ calcd for C₂₂H₁₉BrFN₃O₂: 456.0717; found: 456.0711; HPLC Purity: 97.19%.

### Example 3. General Procedure for Compounds 16 and 17

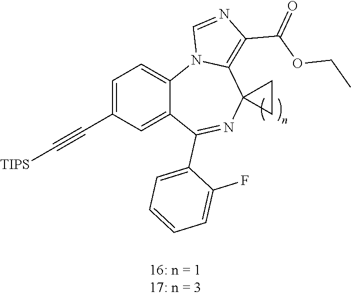

A three stopper RB flask was purged with vacuum and nitrogen 3×. Anhydrous acetonitrile (7.3 mL) was added to the flask and the solvent was degassed with nitrogen. Palladium acetate (28.1 mg, 0.125 mmol) was added followed by the addition of tri(o-tolyl)phosphine (75.7 mg, 0.25 mmol) and the mixture was stirred at room temperature for 30 min. Compound 11 (1.20 g, 2.49 mmol) was added followed by the addition of triethyl amine (0.69 mL, 4.98 mmol), TIPS acetylene (0.67 mL, 2.99 mmol), and additional nitrogen degassed acetonitrile (9.75 mL). The reaction was heated to 75° C. for 4 hr. Upon completion by TLC (100% EtOAc), silica gel (600 mg) was added to the reaction and the reaction was cooled to room temperature while stirring for 30 min. The mixture was filtered over Celite® and washed with acetonitrile. The solvent was removed under reduced pressure and the residue was dissolved in dichloromethane (50 mL) before the organic layer was washed with 5% aqueous sodium bicarbonate (50 mL), followed by 10% aqueous NaCl (50 mL). The organic layer was dried with MgSO4, and the solvent was removed under reduced pressure. The residue was loaded onto a precolumn with chloroform and separated by Biotage: 5-55% ethyl acetate in hexanes (20 CV), 55-90% ethyl acetate in hexanes (5 CV). The desired product 17 was obtained as a yellow solid (1.32 g, 91.2%): ¹H NMR (500 MHz, CDCl3) δ 7.89 (s, 1H), 7.67-7.65 (dd, J=3.38, 1.85 Hz, 1H), 7.54-7.49 (m, 2H), 7.44-7.40 (m, 1H), 7.29-7.28 (m, 1H), 7.24-7.21 (dt, J=3.22, 1.05 Hz, 1H), 7.06-7.02 (m, 1H), 4.40-4.35 (q, J=7.14 Hz, 2H), 2.83-2.81 (m, 1H), 2.67-2.61 (m, 1H), 1.90 (m, 2H), 1.72-1.57 (m, 4H), 1.41-1.38 (t, J=7.13 Hz, 3H), 1.11-1.09 (m, 21H); ¹³C NMR (126 MHz, CDCl3) δ 163.78 (s), 161.50 (s), 160.32 (d, 1JCF=251.19 Hz), 140.82 (s), 135.42 (d, J=5.51 Hz), 135.26 (d, J=8.58 Hz), 135.01 (d, J=12.35 Hz), 132.56 (s), 131.79 (d, 3JCF=7.41 Hz), 131.35 (d, 3JCF=9.54 Hz), 130.41 (s), 129.91 (s), 128.22 (d, 2JCF=12.32 Hz), 124.35 (d, 3JCF=3.29 Hz), 123.02 (s), 122.51 (m), 116.13 (m), 104.62 (s), 94.00 (s), 67.34 (s), 61.18 (t, J=12.41 Hz), 40.97 (t, J=17.85 Hz), 35.86 (t, J=15.68 Hz), 23.42 (m), 18.56 (m), 14.26 (m), 11.18 (m); ¹⁹F NMR (471 MHz, CDCl3) δ −112.39; HRMS (ESI/Q-TOF): m/z [M+H]+ calcd for C₃₅H₄₂FN₃O₂Si: 584.31031; found: 584.30819; HPLC Purity: 98.46%.

### Example 4. General Procedure for Compounds 18 and 19

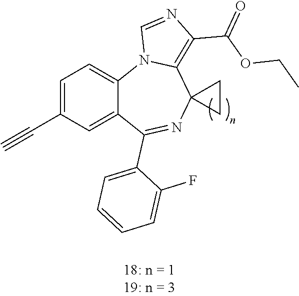

Compound 16 (939.69 mg, 1.69 mmol) was dissolved in a solution of THF (9.32 mL) and H₂O (93.2 μL). The mixture was cooled to −20° C. in a dry ice/IPA bath before 1M TBAF in THF (1.94 mL) was added dropwise over 5 min. The reaction was then warmed to room temperature and stirred for 1.5 hr upon which all the starting material had been consumed by TLC (100% EtOAc:Hex). The reaction was then diluted with ethyl acetate (60 mL), and the organic layer was washed with 10% aqueous NaCl (60 mL, 2×). The organic layer was dried with MgSO4, and the solvent was removed under reduced pressure. The residue was loaded onto a precolumn with chloroform and separated by Biotage: 35-90% ethyl acetate in hexanes (25 CV). The desired product 18 was obtained as an off-white solid (666.2 mg, 98.6%): ¹H NMR (500 MHz, CDCl3) δ 7.95 (s, 1H), 7.76-7.74 (dd, J=3.37, 1.80 Hz, 1H), 7.60-7.58 (m, 2H), 7.48-7.43 (m, 1H), 7.42-7.41 (d, J=1.60 Hz, 1H), 7.25-7.22 (dt, J=3.23, 1.00 Hz, 1H), 7.06-7.02 (m, 1H), 4.50-4.35 (m, 2H), 3.17 (s, 1H), 2.07-2.03 (m, 1H), 1.74-1.71 (m, 1H), 1.46-1.43 (t, J=7.13 Hz, 3H), 0.78-0.68 (m, 2H); ¹³C NMR (126 MHz, CDCl3) δ 168.35 (s), 162.25 (s), 160.22 (d, 1JCF=251.55 Hz), 140.26 (s), 135.20 (d, 3JCF=6.10 Hz), 134.88 (s), 134.18 (d, 3JCF=8.90 Hz), 133.47 (s), 132.35 (d, 3JCF=8.38 Hz), 131.24 (s), 131.01 (d, 4JCF=1.39 Hz), 129.13 (s), 127.27 (d, 2JCF=11.94 Hz), 124.44 (d, 3JCF=3.47 Hz), 122.43 (s), 121.66 (s), 116.24 (d, 2JCF=21.59 Hz), 81.54 (d, J=2.25 Hz), 79.63 (d, J=10.29 Hz), 60.83 (t, J=7.01 Hz), 37.26 (s), 15.11 (s), 14.46 (d, J=8.23 Hz); ¹⁹F NMR (471 MHz, CDCl3) δ −111.82-−111.87 (qu, J=5.59 Hz); HRMS (ESI/Q-TOF): m/z [M+H]+ calcd for C₂₄H₁₈FN₃O₂: 400.14558; found: 400.14606; HPLC Purity: 97.27%.

### Example 5. General Procedure for Compounds 22 and 23

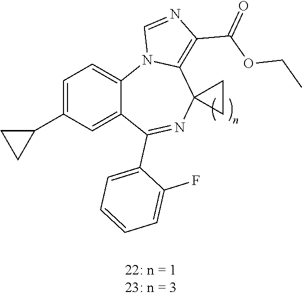

A mixture of toluene (11.2 mL) and water (1.62 mL) was degassed with nitrogen before compound 11 (560.2 mg, 1.16 mmol) was added. Cyclopropyl boronic acid (498.8 mg, 5.81 mmol) was then added, followed by potassium phosphate (1.06 g, 4.99 mmol), palladium acetate (26.1 mg, 0.12 mmol), and tri(o-tolyl)phosphine (70.7 mg, 0.23 mmol). The reaction was then heated to 100° C. for 18 hr before being cooled back to room temperature and H₂O (50 mL) was added. The aqueous layer was extracted with ethyl acetate (50 mL, 3×) and the organic layers were combined before being washed with brine (150 mL) and dried with MgSO4. The solvent was removed under reduced pressure and the residue was loaded onto a precolumn with chloroform and separated by Biotage: 25-95% ethyl acetate in hexanes (20 CV). The desired product 23 was obtained as an off-white solid (331.6 mg, 64.4%): ¹H NMR (500 MHz, CDCl3) −20° C. δ 7.93 (s, 1H), 7.55-7.53 (m, 1H), 7.48-7.46 (m, 1H), 7.45-7.42 (m, 1H), 7.26-7.23 (dt, J=3.14, 0.70 Hz, 1H), 7.19-7.17 (dd, J=3.35, 1.75 Hz, 1H), 7.06-7.03 (m, 1H), 6.97-6.96 (m, 1H), 4.39-4.34 (q, J=7.12 Hz, 2H), 2.87-2.85 (m, 1H), 2.63-2.56 (m, 1H), 1.92-1.90 (m, 2H), 1.88-1.83 (m, 1H), 1.75-1.70 (m, 1H), 1.66-1.52 (m, 3H), 1.41-1.38 (t, J=7.15 Hz, 3H), 1.05-1.02 (m, 2H), 0.72-0.62 (m, 2H); ¹³C NMR (126 MHz, CDCl3) −20° C. δ 163.78 (s), 162.85 (s), 160.26 (d, 1JCF=251.03 Hz), 144.26 (s), 141.20 (s), 135.45 (s), 132.87 (s), 132.00 (d, 3JCF=6.81 Hz), 131.58 (s), 129.28 (s), 129.07 (s), 128.47 (d, 2JCF=15.49 Hz), 127.99 (s), 127.30 (s), 124.64 (d, 3JCF=3.15 Hz), 122.69 (s), 116.28 (d, 2JCF=21.32 Hz), 67.16 (s), 61.42 (s), 41.19 (s), 35.57 (s), 23.78 (s), 23.15 (s), 15.20 (s), 14.49 (s), 10.70 (s); ¹⁹F NMR (471 MHz, CDCl3) −20° C. δ −112.29; HRMS (ESI/Q-TOF): m/z [M+H]+ calcd for C₂₇H₂₆FN₃O₂: 444.20818; found: 444.20450; HPLC Purity: 98.93%.

### Example 6. Synthesis of 8-ethynyl-6-(2-fluorophenyl)spiro[benzo[f]imidazo[1,5-a][1,4]diazepine-4,1′-cyclopropane]-3-carboxylic acid (20)

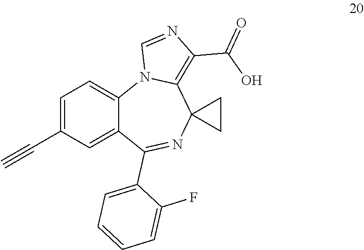 [SMILES: C#Cc1ccc2c(c1)C(c1ccccc1F)=NC1(CC1)c1c(C(=O)O)ncn1-2]

Compound 18 (186.89 mg, 0.41 mmol) was dissolved in tetrahydrofuran (13 mL) and cooled to 0° C. Solid sodium hydroxide was added (493.6 mg, 12.34 mmol), followed by the addition of H₂O (307 μL). The reaction was then removed from the ice bath and gently heated to 80° C. for 18 hr. When all of the starting material had dropped to the baseline via TLC (100% EtOAc), the reaction was cooled to room temperature. Acetic acid was added until the pH was observed to be ˜5, and the reaction was then cooled to 50° C. and allowed to stir for 20 hr. The reaction was then concentrated to dryness under reduced pressure. The residue was dissolved in H₂O (3.5 mL) and portioned into 0.5 mL fractions. To each fraction was added an additional 1 mL of H₂O causing the desired product to precipitate out of solution. The fractions were centrifuged and the solution was decanted. The solid fractions were combined and washed with an additional 6 mL of H₂O to remove any residual acetic acid. The title compound was then collected by filtration to yield a white powder. No further purification was conducted. (140.57 mg, 80.2%): ¹H NMR (500 MHz, d₆-DMSO) δ 8.26 (s, 1H), 7.89-7.86 (dd, J=3.62, 2.25 Hz, 1H), 7.77-7.75 (d, J=8.65 Hz, 1H), 7.49-7.45 (m, 2H), 7.25-7.22 (m, 2H), 7.16-7.12 (m, 1H), 1.78 (m, 1H), 1.34 (m, 1H), 0.61 (m, 2H); ¹³C NMR (126 MHz, d₆-DMSO) δ 167.26 (s), 164.18 (s), 159.91 (d, 1JCF=248.64 Hz), 135.49 (s), 135.35 (s), 134.50 (s), 133.13 (d, 3JCF=8.46 Hz), 132.07 (d, 3JCF=4.73 Hz), 131.94 (d, 4JCF=1.62 Hz), 127.43 (d, 2JCF=12.05 Hz), 125.84 (s), 125.16 (d, 3JCF=3.13 Hz), 120.03 (s), 116.48 (d, 2JCF=21.29 Hz), 37.64 (s), 31.78 (S), 14.80 (s), 14.40 (s); ¹⁹F NMR (471 MHz, d₆-DMSO) δ −113.54-−113.59 (qu, J=5.84 Hz); HRMS (ESI/Q-TOF): m/z [M+H]+ calcd for C₂₀H₁₃BrFN₃O₂: 426.02479; found: 426.02602; HPLC Purity: 99.96%.

### Example 7. Synthesis of 8-ethynyl-6-(2-fluorophenyl)spiro[benzo[f]imidazo[1,5-a][1,4]diazepine-4,1′-cyclopentane]-3-carboxylic acid (21)

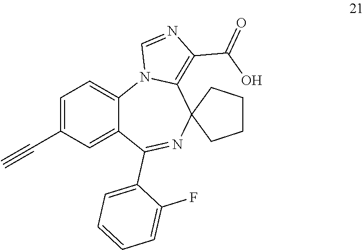 [SMILES: C#Cc1ccc2c(c1)C(c1ccccc1F)=NC1(CCCC1)c1c(C(=O)O)ncn1-2]

Compound 19 (529.1 mg, 1.24 mmol) was dissolved in tetrahydrofuran (37.5 mL) and cooled to 0° C. Solid sodium hydroxide was added (1.49 g, 37.12 mmol), followed by the addition of H₂O (889.7 μL). The reaction was then removed from the ice bath and gently heated to 80° C. for 18 hr. When all of the starting material had dropped to the baseline via TLC (100% EtOAc), the reaction was cooled to room temperature. Acetic acid was added until the pH was observed to be ˜5, and the reaction was then cooled to 50° C. and allowed to stir for 20 hr. The reaction was then concentrated to dryness under reduced pressure. The residue was dissolved in H₂O (6 mL) and portioned into 0.5 mL fractions. To each fraction was added an additional 1 mL of H₂O causing the desired product to precipitate out of solution. The fractions were centrifuged and the solution was decanted. The solid fractions were combined and washed with an additional 15 mL of H₂O to remove any residual acetic acid. The title compound was then collected by filtration to yield a white powder. No further purification was conducted. (276.8 mg, 56.0%): ¹H NMR (500 MHz, d₆-DMSO) δ 8.27 (s, 1H), 7.89-7.87 (d, J=8.4 Hz, 1H), 7.81 (dd, J=3.40, 1.85 Hz, 1H), 7.56-7.50 (m, 2H), 7.34-7.30 (dt, J=3.17, 0.85 Hz, 1H), 7.24-7.23 (m, 1H), 7.20-7.19 (m, 1H), 4.35 (s, 1H), 2.55 (m, 1H), 1.77 (m, 2H), 1.62-1.48 (m, 5H); ¹³C NMR (126 MHz, d₆-DMSO) δ 167.09 (s), 161.01 (s), 159.98 (d, 1JCF=248.03 Hz), 136.02 (s), 135.75 (s), 135.48 (s), 132.68 (s), 132.55 (d, 3JCF=8.40 Hz), 131.98 (d, 4JCF=2.19 Hz), 130.05 (s), 128.64 (d, 2JCF=12.63 Hz), 125.14 (d, 3JCF=3.09 Hz), 124.26 (s), 120.80 (s), 116.40 (d, 2JCF=21.29 Hz), 83.25 (s), 82.14 (s), 67.12 (s), 36.09 (s), 23.56 (s); ¹⁹F NMR (471 MHz, d₆-DMSO) δ −114.22; HRMS (ESI/Q-TOF): m/z [M+H]+ calcd for C₂₄H₁₈FN₃O₂: 400.14558; found: 400.14649; HPLC Purity: 99.71%.

### Example 8. Synthesis of 8-cyclopropyl-6-(2-fluorophenyl)spiro[benzo[f]imidazo[1,5-a][1,4]diazepine-4,1′-cyclopropane]-3-carboxylic acid (24)

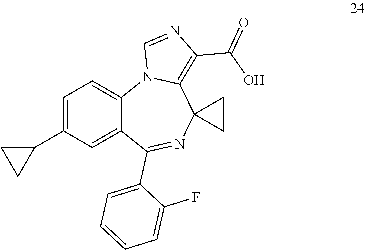 [SMILES: O=C(O)c1ncn2c1C1(CC1)N=C(c1ccccc1F)c1cc(C3CC3)ccc1-2]

Compound 22 (236.18 g, 0.57 mmol) was dissolved in tetrahydrofuran (16.7 mL) and cooled to 0° C. Solid sodium hydroxide was added (682.2 mg, 17.05 mmol), followed by the addition of H₂O (397.4 μL). The reaction was then removed from the ice bath and gently heated to 80° C. for 18 hr. When all of the starting material had dropped to the baseline via TLC (100% EtOAc), the reaction was cooled to room temperature. Acetic acid was added until the pH was observed to be ˜5, and the reaction was then cooled to 50° C. and allowed to stir for 20 hr. The reaction was then concentrated to dryness under reduced pressure. The residue was dissolved in H₂O (3.5 mL) and portioned into 0.5 mL fractions. To each fraction was added an additional 1 mL of H₂O causing the desired product to precipitate out of solution. The fractions were centrifuged and the solution was decanted. The solid fractions were combined and washed with an additional 6 mL of H₂O to remove any residual acetic acid. The title compound was then collected by filtration to yield a white powder. No further purification was conducted. (151.45 g, 68.7%): ¹H NMR (500 MHz, d₆-DMSO) δ 8.30 (s, 1H), 7.76-7.74 (d, J=8.35 Hz, 1H), 7.57-7.52 (m, 2H), 7.36-7.34 (dd, J=3.50, 2.05 Hz, 1H), 7.33-7.30 (dt, J=3.21, 0.95 Hz, 1H), 7.23-7.19 (m, 1H), 6.96-6.95 (m, 1H), 1.97-1.92 (m, 1H), 1.84 (m, 1H), 1.43-1.42 (m, 1H), 0.97-0.96 (m, 2H), 0.67-0.62 (in, 4H); ¹³C NMR (126 MHz, d₆-DMSO) δ 168.73 (s), 164.11 (s), 159.95 (d, 1JCF=248.61 Hz), 143.59 (s), 139.49 (s), 135.45 (s), 132.77 (d, 3JCF=8.33 Hz), 132.64 (s), 132.58 (s), 131.84 (d, 4JCF=2.26 Hz), 130.01 (s), 128.47 (s), 128.07 (d, 3JCF=12.26 Hz), 127.21 (s), 126.32 (d, 2JCF=12.40 Hz), 125.02 (d, 3JCF=2.98 Hz), 123.51 (s), 116.38 (d, 2JCF=21.42 Hz), 37.48 (s), 15.06 (s), 14.69 (d, J=53.58 Hz), 10.51 (d, J=32.82 Hz); ¹⁹F NMR (471 MHz, d₆-DMSO) δ −113.51; HRMS (ESI/Q-TOF): m/z [M+H]+ calcd for C₂₃H₁₈FN₃O₂: 388.14558; found: 388.14551; HPLC Purity: 99.49%.

### Example 9. Synthesis of 8-cyclopropyl-6-(2-fluorophenyl)spiro[benzo[f]imidazo[1,5-a][1,4]diazepine-4,1′-cyclopentane]-3-carboxylic acid (25)

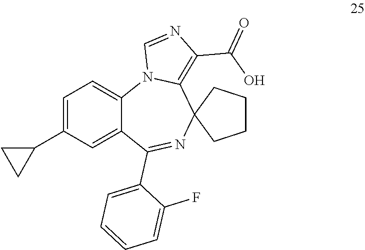 [SMILES: O=C(O)c1ncn2c1C1(CCCC1)N=C(c1ccccc1F)c1cc(C3CC3)ccc1-2]

Compound 23 (196.2 g, 0.44 mmol) was dissolved in tetrahydrofuran (13.9 mL) and cooled to 0° C. Solid sodium hydroxide was added (0.53 mg, 13.27 mmol), followed by the addition of H₂O (330 μL). The reaction was then removed from the ice bath and gently heated to 80° C. for 18 hr. When all of the starting material had dropped to the baseline via TLC (100% EtOAc), the reaction was cooled to room temperature. Acetic acid was added until the pH was observed to be ˜5, and the reaction was then cooled to 50° C. and allowed to stir for 20 hr. The reaction was then concentrated to dryness under reduced pressure. The residue was dissolved in H₂O (3.5 mL) and portioned into 0.5 mL fractions. To each fraction was added an additional 1 mL of H₂O causing the desired product to precipitate out of solution. The fractions were centrifuged and the solution was decanted. The solid fractions were combined and washed with an additional 6 mL of H₂O to remove any residual acetic acid. The product title compound 25 was then collected by filtration to yield a white powder. No further purification was conducted. (121.0 mg, 65.8%): ¹H NMR (500 MHz, d₆-DMSO) δ 8.22 (s, 1H), 7.73-7.71 (d, J=8.35, 1H), 7.55-7.49 (m, 2H), 7.32-7.29 (m, 2H), 7.23-7.20 (m, 1H), 6.93-6.92 (m, 1H), 2.56 (m, 1H), 2.48 (m, 1H), 1.96-1.91 (m, 1H), 1.77 (m, 2H), 1.61-1.47 (m, 4H), 0.96-0.95 (m, 2H), 0.65-0.63 (m, 2H); ¹³C NMR (126 MHz, d₆-DMSO) δ 166.62 (s), 162.04 (s), 160.05 (d, 1JCF=248.18 Hz), 143.65 (s), 137.59 (s), 136.05 (s), 135.89 (s), 133.13 (s), 132.35 (s), 131.91 (s), 129.61 (s), 128.97 (d, 3JCF=12.54 Hz), 128.42 (s), 127.03 (d, 3JCF=13.99 Hz), 125.03 (d, 3JCF=3.92 Hz), 123.72 (d, 2JCF=15.89 Hz), 116.35 (d, 2JCF=21.26 Hz), 67.00 (s), 35.74 (s), 23.54 (s), 15.07 (s), 14.95 (s), 10.63 (s), 10.30 (s); ¹⁹F NMR (471 MHz, d₆-DMSO) δ −114.06; HRMS (ESI/Q-TOF): m/z [M+H]+ calcd for C₂₅H₂₂FN₃O₂: 416.17688; found: 416.17786; HPLC Purity: 99.67%.

### Example 10. Synthesis of 8-bromo-6-(2-fluorophenyl)spiro[benzo[f]imidazo[1,5-a][1,4]diazepine-4,1′-cyclopropane]-3-carboxylic acid (26)

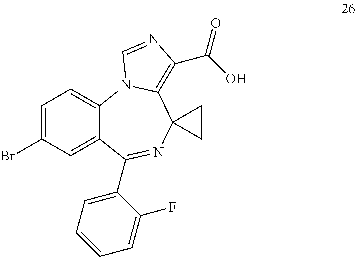 [SMILES: O=C(O)c1ncn2c1C1(CC1)N=C(c1ccccc1F)c1cc(Br)ccc1-2]

Compound 9 (186.89 mg, 0.41 mmol) was dissolved in tetrahydrofuran (13 mL) and cooled to 0° C. Solid sodium hydroxide was added (493.6 mg, 12.34 mmol), followed by the addition of H₂O (307 μL). The reaction was then removed from the ice bath and gently heated to 80° C. for 18 hr. When all of the starting material had dropped to the baseline via TLC (100% EtOAc), the reaction was cooled to room temperature. Acetic acid was added until the pH was observed to be ˜5, and the reaction was then cooled to 50° C. and allowed to stir for 20 hr. The reaction was then concentrated to dryness under reduced pressure. The residue was dissolved in H₂O (3.5 mL) and portioned into 0.5 mL fractions. To each fraction was added an additional 1 mL of H₂O causing the desired product to precipitate out of solution. The fractions were centrifuged and the solution was decanted. The solid fractions were combined and washed with an additional 6 mL of H₂O to remove any residual acetic acid. The title compound was then collected by filtration to yield a white powder. No further purification was conducted. (140.57 mg, 80.2%): ¹H NMR (500 MHz, d₆-DMSO) δ 8.26 (s, 1H), 7.89-7.86 (dd, J=3.62, 2.25 Hz, 1H), 7.77-7.75 (d, J=8.65 Hz, 1H), 7.49-7.45 (m, 2H), 7.25-7.22 (m, 2H), 7.16-7.12 (m, 1H), 1.78 (m, 1H), 1.34 (m, 1H), 0.61 (m, 2H); ¹³C NMR (126 MHz, d₆-DMSO) δ 167.26 (s), 164.18 (s), 159.91 (d, ¹JCF=248.64 Hz), 135.49 (s), 135.35 (s), 134.50 (s), 133.13 (d, ³JCF=8.46 Hz), 132.07 (d, ³J_{CF}=4.73 Hz), 131.94 (d, ⁴J_{CF}=1.62 Hz), 127.43 (d, ²J_{CF}=12.05 Hz), 125.84 (s), 125.16 (d, ¹J_{CF}=3.13 Hz), 120.03 (s), 116.48 (d, ²J_{CF}=21.29 Hz), 37.64 (s), 31.78 (S), 14.80 (s), 14.40 (s); ¹⁹F NMR (471 MHz, d₆-DMSO) δ −113.54-−113.59 (qu, J=5.84 Hz); HRMS (ESI/Q-TOF): m/z [M+H]⁺ calcd for C₂₀H₁₃BrFN₃O₂: 426.02479; found: 426.02602; HPLC Purity: 99.96%.

### Example 11. Synthesis of 8-bromo-6-(2-fluorophenyl)spiro[benzo[f]imidazo[1,5-a][1,4]diazepine-4,1′-cyclobutane]-3-carboxylic acid (27)

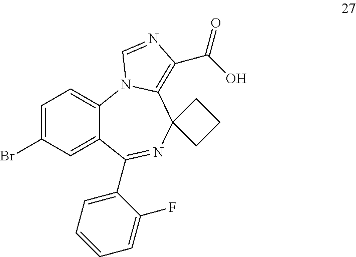 [SMILES: O=C(O)c1ncn2c1C1(CCC1)N=C(c1ccccc1F)c1cc(Br)ccc1-2]

Compound 10 (158.89 mg, 0.34 mmol) was dissolved in tetrahydrofuran (11.3 mL) and cooled to 0° C. Solid sodium hydroxide was added (407.1 mg, 10.18 mmol), followed by the addition of H₂O (260 μL). The reaction was then removed from the ice bath and gently heated to 80° C. for 18 hr. When all of the starting material had dropped to the baseline via TLC (100% EtOAc), the reaction was cooled to room temperature. Acetic acid was added until the pH was observed to be ˜5, and the reaction was then cooled to 50° C. and allowed to stir for 20 hr. The reaction was then concentrated to dryness under reduced pressure. The residue was dissolved in H₂O (3.5 mL) and portioned into 0.5 mL fractions. To each fraction was added an additional 1 mL of H₂O causing the desired product to precipitate out of solution. The fractions were centrifuged and the solution was decanted. The solid fractions were combined and washed with an additional 6 mL of H₂O to remove any residual acetic acid. The product was then collected by filtration to yield a white powder. The solid was slurried in 5 mL of 5 M HCl at 95° C. for 18 hr upon which the diazepine ring had been completely broken by TLC (1.8 mL MeOH+0.1 mL AcOH+4.3 mL chloroform). The mixture was then cooled to room temperature and the product was collected by filtration. The solid material was then washed with hot IPA (1 mL, 2×) and the product was again filtered to yield the hydrochloride salt of the open form of the title compound, 1-(1-(4-bromo-2-(2-fluorobenzoyl)phenyl)-4-carboxy-1H-imidazol-5-yl)cyclobutan-1-aminium chloride. (92.78 mg, 62.1%): ¹H NMR (500 MHz, d₆-DMSO) δ 8.54 (s, 1H), 7.99-7.96 (dd, J=3.63, 2.25 Hz, 1H), 7.88-7.86 (d, J=2.25 Hz, 1H), 7.68-7.65 (dt, J=3.40, 1.70 Hz, 1H), 7.60-7.55 (m, 1H), 7.40-7.39 (m, 1H), 7.37-7.34 (dt, J=3.21, 1.00 Hz, 1H), 7.26-7.22 (m, 1H), 3.51-3.46 (m, 1H), 2.82-2.76 (m, 1H), 2.07-2.02 (m, 1H), 1.88-1.79 (m, 2H), 1.69-1.62 (m, 1H); ¹³C NMR (126 MHz, d₆-DMSO) δ 165.08 (s), 160.39 (s), 157.71 (d, 1JCF=336.58 Hz), 139.95 (s), 135.99 (s), 135.48 (s), 134.24 (s), 132.95 (d, 3JCF=8.24 Hz), 132.17 (d, 4JCF=1.57 Hz), 131.94 (s), 131.75 (s), 127.69 (d, 2JCF=12.05 Hz), 125.87 (s), 125.19 (d, 3JCF=3.51 Hz), 120.32 (s), 118.91 (s), 117.86 (s), 116.47 (d, 2JCF=21.27 Hz), 61.99 (s), 35.91 (s), 34.56 (s), 15.87 (s); ¹⁹F NMR (471 MHz, d₆-DMSO) δ −113.78-−113.83 (qu, J=5.69 Hz); HRMS (ESI/Q-TOF): m/z [M+H]+ calcd for C₂₁H₁₇BrFN₃O₃: 458.05101; found: 458.05074; HPLC Purity: 99.67%.

For the closed form 8-bromo-6-(2-fluorophenyl)spiro[benzo[f]imidazo[1,5-a][1,4]diazepine-4,1′-cyclobutane]-3-carboxylic acid: ¹H NMR (500 MHz, d₆-DMSO) δ 8.27 (s, 1H), 8.01-7.99 (dd, J=3.63, 2.27 Hz, 1H), 7.65-7.64 (m, 1H), 7.59-7.58 (m, 1H), 7.39-7.38 (d, J=1.65 Hz, 1H), 7.34-7.33 (d, J=0.95 Hz, 1H), 7.20-7.18 (dd, J=3.13, 1.47 Hz, 2H), 6.89-6.86 (dt, J=4.07, 1.12 Hz, 1H), 3.74-3.70 (m, 2H), 3.64-3.60 (m, 2H), 3.57-3.54 (m, 2H); ¹³C NMR (126 MHz, d₆-DMSO) δ 174.41 (s), 160.03 (d, ¹J_{CF}=248.51 Hz), 139.94 (s), 136.08 (s), 135.59 (s), 133.98 (s), 133.57 (s), 133.12 (s), 131.66 (s), 127.71 (d, ³J_{CF}=13.99 Hz), 126.56 (s), 120.96 (s), 120.03 (s), 118.94 (s), 117.94 (s), 112.78 (s), 61.72 (s), 35.27 (s), 34.10 (s), 15.99 (s).

### Example 12. Synthesis of 8-bromo-6-(2-fluorophenyl)spiro[benzo[f]imidazo[1,5-a][1,4]diazepine-4,1′-cyclopentane]-3-carboxylic acid (28)

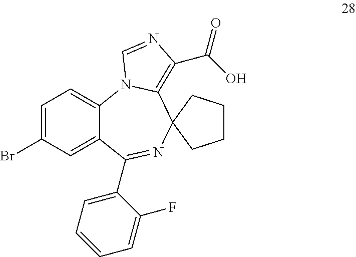 [SMILES: O=C(O)c1ncn2c1C1(CCCC1)N=C(c1ccccc1F)c1cc(Br)ccc1-2]

Compound 11 (286.1 mg, 0.59 mmol) was dissolved in tetrahydrofuran (20.3 mL) and cooled to 0° C. Solid sodium hydroxide was added (712 mg, 17.79 mmol), followed by the addition of H₂O (481 μL). The reaction was then removed from the ice bath and gently heated to 80° C. for 18 hr. When all of the starting material had dropped to the baseline via TLC (100% EtOAc), the reaction was cooled to room temperature. Acetic acid was added until the pH was observed to be ˜5, and the reaction was then cooled to 50° C. and allowed to stir for 20 hr. The reaction was then concentrated to dryness under reduced pressure. The residue was dissolved in H₂O (6 mL) and portioned into 0.5 mL fractions. To each fraction was added an additional 1 mL of H₂O causing the desired product to precipitate out of solution. The fractions were centrifuged and the solution was decanted. The solid fractions were combined and washed with an additional 15 mL of H₂O to remove any residual acetic acid. The title compound was then collected by filtration to yield a white powder. No further purification was conducted. (125.4 mg, 46.5%): ¹H NMR (500 MHz, d₆-DMSO) δ 8.25 (s, 1H), 7.92-7.90 (dd, J=3.57, 2.11 Hz, 1H), 7.83-7.81 (d, J=8.65 Hz, 1H), 7.56-7.51 (m, 2H), 7.33-7.32 (d, J=7.40 Hz, 1H), 7.30-7.29 (m, 1H), 7.24-7.21 (dt, J=6.29, 1.41 Hz, 1H), 2.55 (m, 2H), 1.77 (m, 2H), 1.61 (m, 3H), 1.48 (m, 1H); ¹³C NMR (126 MHz, d₆-DMSO) δ 167.27 (S), 160.47 (S), 159.99 (d, ¹J_{CF}=248.16 Hz), 136.33 (S), 135.93 (S), 135.36 (S), 135.03 (S), 133.47 (S), 132.65 (d, ³J_{CF}=8.15 Hz), 132.01 (d, ⁴J_{CF}=1.38 Hz), 131.84 (S), 131.63 (S), 128.42 (d, ²J_{CF}=12.50 Hz), 125.94 (S), 125.15 (d, ⁴J_{CF}=2.64 Hz), 119.93 (S), 116.43 (d, ²J_{CF}=21.39 Hz), 67.17 (S), 36.16 (S), 23.61 (S); ¹⁹F NMR (471 MHz, d₆-DMSO) δ −114.11; HRMS (ESI/Q-TOF): m/z [M+H]⁺ calcd for C₂₂H₁₇BrFN₃O₂: 454.05609; found: 454.05775; HPLC Purity: 99.48%.

### Example 13. Synthesis of 8-bromo-6-(2-fluorophenyl)spiro[benzo[f]imidazo[1,5-a][1,4]diazepine-4,1′-cyclohexane]-3-carboxylic acid (29)

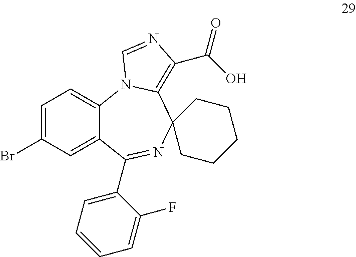 [SMILES: O=C(O)c1ncn2c1C1(CCCCC1)N=C(c1ccccc1F)c1cc(Br)ccc1-2]

Compound 12 (168.93 mg, 0.34 mmol) was dissolved in tetrahydrofuran (12 mL) and cooled to 0° C. Solid sodium hydroxide was added (432.8 mg, 10.82 mmol), followed by the addition of H₂O (284 μL). The reaction was then removed from the ice bath and gently heated to 80° C. for 18 hr. When all of the starting material had dropped to the baseline via TLC (100% EtOAc), the reaction was cooled to room temperature. Acetic acid was added until the pH was observed to be ˜5, and the reaction was then cooled to 50° C. and allowed to stir for 20 hr. The reaction was then concentrated to dryness under reduced pressure. The residue was dissolved in H₂O (4 mL) and portioned into 0.5 mL fractions. To each fraction was added an additional 1 mL of H₂O causing the desired product to precipitate out of solution. The fractions were centrifuged, and the solution was decanted. The solid fractions were combined and washed with an additional 6 mL of H₂O to remove any residual acetic acid. The title compound was then collected by filtration to yield a white powder. No further purification was conducted. (143.2 mg, 89.8%): ¹H NMR (500 MHz, d₆-DMSO) δ 8.07 (s, 1H), 7.76-7.74 (dd, J=3.63, 2.25 Hz, 1H), 7.65-7.64 (d, J=8.65 Hz, 1H), 7.43-7.39 (m, 2H), 7.21-7.18 (dt, J=3.20, 0.95 Hz, 1H), 7.15-7.14 (d, J=2.15 Hz, 1H), 7.13-7.09 (m, 1H), 2.24 (m, 2H), 1.89 (m, 1H), 1.73 (m, 1H), 1.52-1.49 (m, 2H), 1.14-1.10 (m, 4H); ¹³C NMR (126 MHz, d₆-DMSO) δ 168.45 (s), 160.01 (d, ¹J_{CF}=248.17 Hz), 159.67 (s), 135.91 (s), 135.41 (s), 135.29 (s), 132.55 (d, ³J_{CF}=8.31 Hz), 131.95 (d, ⁴J_{CF}=2.57 Hz), 131.71 (s), 130.69 (s), 129.00 (d, ²J_{CF}=12.70 Hz), 125.47 (s), 125.21 (d, ³J_{CF}=3.05 Hz), 119.70 (s), 116.51 (d, ²J_{CF}=21.37 Hz), 59.21 (s), 37.24 (s), 31.72 (s), 25.68 (s), 21.96 (s); ¹⁹F NMR (471 MHz, d₆-DMSO) δ −114.14; HRMS (ESI/Q-TOF): m/z [M+H]⁺ calcd for C₂₃H₁₉BrFN₃O₂: 468.07174; found: 467.07357; HPLC Purity: 99.78%.

### Example 14. Synthesis of 8-chloro-6-(2-fluorophenyl)spiro[benzo[f]imidazo[1,5-a][1,4]diazepine-4,1′-cyclopropane]-3-carboxylic acid (30)

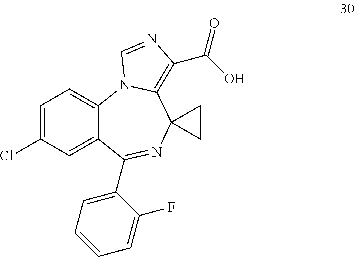 [SMILES: O=C(O)c1ncn2c1C1(CC1)N=C(c1ccccc1F)c1cc(Cl)ccc1-2]

Compound 13 (210.1 mg, 0.51 mmol) was dissolved in tetrahydrofuran (14.9 mL) and cooled to 0° C. Solid sodium hydroxide was added (615.2 mg, 15.38 mmol), followed by the addition of H₂O (353.5 μL). The reaction was then removed from the ice bath and gently heated to 80° C. for 18 hr. When all of the starting material had dropped to the baseline via TLC (100% EtOAc), the reaction was cooled to room temperature. Acetic acid was added until the pH was observed to be ˜5, and the reaction was then cooled to 50° C. and allowed to stir for 20 hr. The reaction was then concentrated to dryness under reduced pressure. The residue was dissolved in H₂O (4 mL) and portioned into 0.5 mL fractions. To each fraction was added an additional 1 mL of H₂O causing the desired product to precipitate out of solution. The fractions were centrifuged and the solution was decanted. The solid fractions were combined and washed with an additional 6 mL of H₂O to remove any residual acetic acid. The title compound was then collected by filtration to yield a white powder. No further purification was conducted. (172.6 mg, 88.2%): ¹H NMR (500 MHz, d₆-DMSO) δ 8.40 (s, 1H), 7.95-7.94 (d, J=8.65 Hz, 1H), 7.87-785 (dd, J=3.68, 2.40 Hz, 1H), 7.59-7.55 (m, 2H), 7.35-7.31 (m, 1H), 7.25-7.21 (m, 2H), 1.85-1.80 (m, 1H), 1.49-1.45 (m, 1H), 0.75-0.69 (m, 2H); ¹³C NMR (126 MHz, d₆-DMSO) δ 167.47 (s), 163.88 (s), 159.91 (d, ¹J_{CF}=248.68 Hz), 139.72 (s), 135.84 (d, ³J_{CF}=10.35 Hz), 133.99 (s), 133.20 (s), 132.51 (d, ³J_{CF}=11.56 Hz), 131.96 (s), 131.87 (s), 129.26 (s), 128.96 (s), 127.35 (d, ²J_{CF}=11.96 Hz), 125.71 (d, ²J_{CF}=12.00 Hz), 125.17 (d, ⁴J_{CF}=2.50 Hz), 116.49 (d, ²J_{CF}=21.02 Hz), 37.60 (s), 14.82 (s), 14.41 (s); ¹⁹F NMR (471 MHz, d₆-DMSO) δ −113.53-−113.58 (qu, J=5.69 Hz) HRMS (ESI/Q-TOF): m/z [M+H]⁺ calcd for C₂₀H₁₃ClFN₃O₂: 382.07531; found: 382.07606; HPLC Purity: 99.82%.

### Example 15. Synthesis of 8-chloro-6-(2-fluorophenyl)spiro[benzo[f]imidazo[1,5-a][1,4]diazepine-4,1′-cyclopentane]-3-carboxylic acid (31)

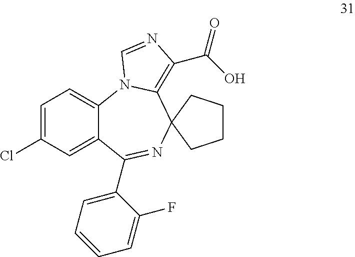 [SMILES: O=C(O)c1ncn2c1C1(CCCC1)N=C(c1ccccc1F)c1cc(Cl)ccc1-2]

Compound 14 (314.2 mg, 0.72 mmol) was dissolved in tetrahydrofuran (22.3 mL) and cooled to 0° C. Solid sodium hydroxide was added (861.0 mg, 21.53 mmol), followed by the addition of H₂O (529.5 μL). The reaction was then removed from the ice bath and gently heated to 80° C. for 18 hr. When all of the starting material had dropped to the baseline via TLC (100% EtOAc), the reaction was cooled to room temperature. Acetic acid was added until the pH was observed to be ˜5, and the reaction was then cooled to 50° C. and allowed to stir for 20 hr. The reaction was then concentrated to dryness under reduced pressure. The residue was dissolved in H₂O (5 mL) and portioned into 0.5 mL fractions. To each fraction was added an additional 1 mL of H₂O causing the desired product to precipitate out of solution. The fractions were centrifuged and the solution was decanted. The solid fractions were combined and washed with an additional 9 mL of H₂O to remove any residual acetic acid. The title compound was then collected by filtration to yield a white powder. No further purification was conducted. (314.9 mg, 84.0%): ¹H NMR (500 MHz, d₆-DMSO) δ 8.32 (s, 1H), 7.93-7.92 (d, J=8.70 Hz, 1H), 7.83-7.81 (dd, J=3.70, 2.45 Hz, 1H), 7.58-7.51 (m, 2H), 7.34-7.31 (dt, J=3.21, 1.00 Hz, 1H), 7.25-7.21 (m, 1H), 7.20-7.19 (d, J=2.40 Hz, 1H), 2.58 (m, 1H), 2.46 (m, 1H), 1.78 (m, 2H), 1.63-1.49 (m, 4H); ¹³C NMR (126 MHz, d₆-DMSO) δ 166.31 (s), 160.77 (s), 160.00 (d, ¹J_{CF}=248.25 Hz), 137.96 (s), 136.59 (s), 136.43 (s), 134.43 (s), 132.55 (d, ²J_{CF}=21.13 Hz), 132.05 (s), 131.42 (s), 131.03 (s), 129.04 (s), 128.27 (d, ³J_{CF}=12.36 Hz), 125.97 (d, ³J_{CF}=3.75 Hz), 125.78 (s), 125.17 (s), 116.46 (d, ²J_{CF}=21.27 Hz), 67.12 (s), 36.00 (s), 23.57 (s); ¹⁹F NMR (471 MHz, d₆-DMSO) δ −114.11; HRMS (ESI/Q-TOF): m/z [M+H]+ calcd for C₂₂H₁₇ClFN₃O₂: 410.10661; found: 410.10755; HPLC Purity: 99.82%.

### Example 16. Synthesis of 6-(2-fluorophenyl)spiro[benzo[f]imidazo[1,5-a][1,4]diazepine-4,1′-cyclopropane]-3-carboxylic acid (32)

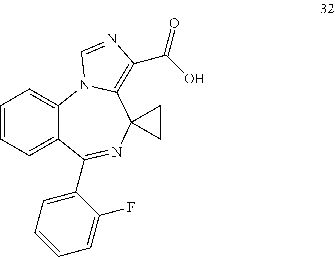 [SMILES: O=C(O)c1ncn2c1C1(CC1)N=C(c1ccccc1F)c1ccccc1-2]

A three stopper RB flask was purged with vacuum and nitrogen 3×. Anhydrous methanol (47 mL) was added, and the solvent was degassed with nitrogen. Sodium bicarbonate (281 mg, 3.35 mmol) was added followed by the addition of 26 (470.62 mg, 1.10 mmol) and 10% palladium on activated carbon (114 mg). A hydrogen balloon was attached to the flask and the flask was gently purged with vacuum and hydrogen 3×. The hydrogen was then allowed to freely flow into the reaction with vigorous stirring for 5 min. The reaction mixture was then filtered over Celite® and washed with methanol. The solvent was removed under reduced pressure and the residue was sonicated in H₂O (2 mL) for 2 minutes. The title compound 32 was then collected by filtration to yield a white powder (313.98 mg, 81.9%): ¹H NMR (500 MHz, d₆-DMSO) δ 8.37 (s, 1H), 7.90-7.88 (d, J=7.90 Hz, 1H), 7.77-7.74 (m, 1H), 7.56-7.52 (m, 2H), 7.50-7.47 (m, 1H), 7.33-7.29 (m, 1H), 7.25-7.18 (m, 2H), 1.86-1.82 (m, 1H), 1.48-1.43 (m, 1H), 0.71-0.67 (m, 1H), 0.63-0.58 (m, 1H); ¹³C NMR (126 MHz, d₆-DMSO) δ 168.80 (s), 164.09 (s), 159.94 (d, 1J_{CF}=248.48 Hz), 139.76 (s), 135.65 (d, 3J_{CF}=19.73 Hz), 135.09 (s), 132.68 (d, 2J_{CF}=20.59 Hz), 131.80 (d, 3J_{CF}=16.10 Hz), 130.26 (s), 130.04 (s), 129.12 (s), 128.07 (d, 3J_{CF}=12.27 Hz), 127.81 (d, 3J_{CF}=12.27 Hz), 125.04 (s), 123.49 (d, 2J_{CF}=22.88 Hz), 116.40 (d, 2J_{CF}=21.17 Hz), 37.48 (s), 14.89 (s), 14.48 (s); ¹⁹F NMR (471 MHz, d₆-DMSO) δ −113.54-−113.59 (qu, J=5.42 Hz); HRMS (ESI/Q-TOF): m/z [M+H]+ calcd for C₂₀H₁₄FN₃O₂: 348.11428; found: 348.11441; HPLC Purity: 99.51%.

### Example 17. Synthesis of 6-(2-fluorophenyl)spiro[benzo[f]imidazo[1,5-a][1,4]diazepine-4,1′-cyclopentane]-3-carboxylic acid (33)

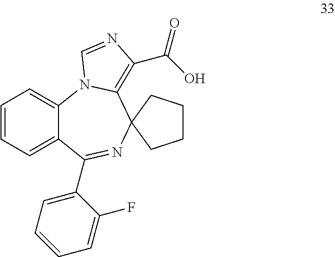 [SMILES: O=C(O)c1ncn2c1C1(CCCC1)N=C(c1ccccc1F)c1ccccc1-2]

A three stopper RB flask was purged with vacuum and nitrogen 3×. Anhydrous methanol (17 mL) was added, and the solvent was degassed with nitrogen. Sodium bicarbonate (102 mg, 1.21 mmol) was added followed by the addition of compound 31 (170.4 mg, 0.38 mmol) and 10% palladium on activated carbon (42 mg). A hydrogen balloon was attached to the flask and the flask was gently purged with vacuum and hydrogen 3×. The hydrogen was then allowed to freely flow into the reaction with vigorous stirring for 5 min. The reaction mixture was then filtered over Celite® and washed with methanol. The solvent was removed under reduced pressure and the residue was sonicated in H₂O (2 mL) for 2 minutes. The product was then collected by filtration to yield a white powder (69.6 mg, 49.4%): ¹H NMR (500 MHz, d₆-DMSO) δ 8.27 (s, 1H), 7.86-7.85 (m, 1H), 7.72-7.70 (m, 1H), 7.55-7.49 (m, 2H), 7.46-7.43 (m, 1H), 7.32-7.29 (m, 1H), 7.22-7.19 (m, 2H), 2.57 (m, 1H), 2.47 (m, 1H), 1.78 (m, 2H), 1.53 (m, 4H); ¹³C NMR (126 MHz, d₆-DMSO) δ 166.69 (s), 162.10 (s), 160.05 (d, ¹J_{CF}=248.05 Hz), 136.16 (s), 135.61 (s), 132.70 (s), 132.33 (d, ³J_{CF}=8.51 Hz), 131.89 (d, ⁴J_{CF}=2.46 Hz), 129.81 (s), 129.01 (d, ²J_{CF}=12.72 Hz), 127.79 (s), 125.05 (d, ³J_{CF}=3.15 Hz), 123.64 (s), 116.36 (d, ²J_{CF}=21.47 Hz), 67.00 (s), 35.76 (s), 23.46 (s); ¹⁹F NMR (471 MHz, d₆-DMSO) δ −114.13; HRMS (ESI/Q-TOF): m/z [M+H]⁺ calcd for C₂₂H₁₈FN₃O₂: 376.14558; found: 376.14589; HPLC Purity: 99.27%.

### Example 18. Synthesis of 8-bromo-6-(2-fluorophenyl)-4,4-dimethyl-4H-benzo[f]imidazo[1,5-a][1,4]diazepine-3-carboxylic acid (34)

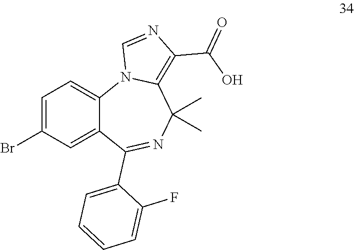 [SMILES: CC1(C)N=C(c2ccccc2F)c2cc(Br)ccc2-n2cnc(C(=O)O)c21]

Compound 15 (202.1 mg, 0.44 mmol) was dissolved in tetrahydrofuran (14.3 mL) and cooled to 0° C. Solid sodium hydroxide was added (531.5 mg, 13.29 mmol), followed by the addition of H₂O (340 μL). The reaction was then removed from the ice bath and gently heated to 80° C. for 18 hr. When all of the starting material had dropped to the baseline via TLC (100% EtOAc), the reaction was cooled to room temperature. Acetic acid was added until the pH was observed to be ˜5, and the reaction was then cooled to 50° C. and allowed to stir for 20 hr. The reaction was then concentrated to dryness under reduced pressure. The residue was dissolved in H₂O (3.5 mL) and portioned into 0.5 mL fractions. To each fraction was added an additional 1 mL of H₂O causing the desired product to precipitate out of solution. The fractions were centrifuged and the solution was decanted. The solid fractions were combined and washed with an additional 6 mL of H₂O to remove any residual acetic acid. The product was then collected by filtration to yield a white powder. The solid was slurried in 5 mL of 5 M HCl at 95° C. for 18 hr upon which the diazepine ring had been completely broken by TLC (1.8 mL MeOH+0.1 mL AcOH+4.3 mL chloroform). The mixture was then cooled to room temperature and the product was collected by filtration. The solid material was then washed with hot IPA (1 mL, 2×) and the product was again filtered to yield the hydrochloride salt of the open form of the title compound, 2-(1-(4-bromo-2-(2-fluorobenzoyl)phenyl)-4-carboxy-1H-imidazol-5-yl)propan-2-aminium chloride. (59.3 mg, 31.2%): ¹H NMR (500 MHz, MeOD) δ 8.05-8.03 (dd, J=3.56-2.25 Hz, 1H), 7.87-7.86 (m, 2H), 7.81-7.79 (m, 1H), 7.77-7.70 (m, 2H), 7.41-7.38 (m, 1H), 7.32-7.28 (m, 1H), 1.77 (s, 3H), 1.53 (s, 3H); ¹³C NMR (126 MHz, MeOD) δ 190.18 (s), 166.65 (s), 161.10 (d, 1JCF=254.49 Hz), 139.80 (s), 139.43 (s), 138.47 (s), 136.11 (d, 3JCF=9.12 Hz), 135.63 (s), 133.04 (s), 132.07 (s), 131.39 (s), 124.88 (d, 3JCF=3.43 Hz), 124.76 (d, 2JCF=11.14 Hz), 124.65 (s), 116.51 (d, 2JCF=21.86 Hz), 52.86 (s), 25.77 (s), 25.63 (s); ¹⁹F NMR (471 MHz, MeOD) δ −110.50, −114.21; HRMS (ESI/Q-TOF): m/z [M+H]+ calcd for C₂₀H₁₇BrFN₃O₃: 446.05101; found: 446.05095; HPLC Purity: 96.12%.

For the closed form 8-bromo-6-(2-fluorophenyl)-4,4-dimethyl-4H-benzo[f]imidazo[1,5-a][1,4]diazepine-3-carboxylic acid: ¹H NMR (500 MHz, d₆-DMSO) δ 7.85-7.84 (dd, J=3.55, 2.25 Hz, 1H), 7.73-772 (d, J=2.08 Hz, 1H), 7.62-7.57 (m, 1H), 7.53-7.50 (dt, J=3.24, 1.25 Hz, 1H), 7.44-7.42 (m, 2H), 7.23-7.19 (m, 2H), 1.76 (s, 3H), 1.09 (s, 3H); ¹³C NMR (126 MHz, d₆-DMSO) δ 190.26 (s), 165.62 (s), 159.90 (d, ¹J_{CF}=247.84 Hz), 139.50 (s), 138.06 (s), 137.22 (d, ³J_{CF}=21.15 Hz), 135.44 (d, ³J_{CF}=21.58 Hz), 135.14 (s), 134.60 (s), 133.06 (s), 132.25 (s), 125.43 (S), 123.33 (s), 117.28 (s), 51.24 (s), 28.35 (s).

### Example 19. Synthesis of 7-bromo-3-ethyl-5-(2-fluorophenyl)-1,3-dihydro-2H-benzo[e][1,4]diazepin-2-one (35)

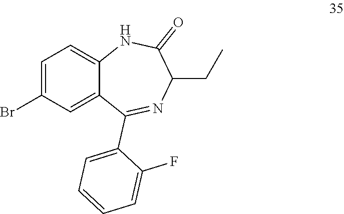 [SMILES: CCC1N=C(c2ccccc2F)c2cc(Br)ccc2NC1=O]

2-Amino-5-bromo-2′-fluorobenzophenone (1.68 g, 5.71 mmol) was added to anhydrous toluene (60 mL), followed by the addition of trifluoroacetic acid (0.87 mL, 11.42 mmol) dropwise over a period of 10 min, and the mixture was allowed to stir at room temperature for 30 min. 4-Ethyloxazolidine-2,5-dione (1.11 g, 8.57 mmol) was added portion wise and the reaction was heated to 50° C. for 24 hr. After the majority of the starting material had been consumed by TLC (50% EtOAc:Hex), triethylamine (1.67 mL, 12.00 mmol) was added dropwise over a period of 15 min at which point fuming was observed in the reaction. The reaction was then heated to 100° C. for 24 hr at which point disappearance of the intermediate was observed via TLC (50% EtOAc:Hex). Upon cooling to room temperature, the solvent was removed under reduced pressure and the residue was dissolved in ethyl acetate (50 mL). The organic layer was washed with 5% aqueous sodium bicarbonate (50 mL), followed by 10% aqueous NaCl (50 mL). The organic layer was then dried with MgSO4 and the solvent was removed under reduced pressure. The residue was stripped with 10% EtOAc:Heptane (20 mL, 2×) followed by a trituration in 10% EtOAc:Heptane (35 mL) at 60° C. for 4 hr. The title compound was collected by filtration to yield a light yellow solid (1.64 g, 79.5%): ¹H NMR (500 MHz, CDCl3) δ 8.96 (s, 1H), 7.52-7.49 (m, 2H), 7.41-7.36 (m, 1H), 7.29-7.28 (d, J=2.05 Hz, 1H), 7.19-7.15 (m, 1H), 7.02-6.97 (m, 2H), 3.40-3.37 (t, J=7.05 Hz, 1H), 2.22-2.16 (m, 2H), 1.04-1.01 (t, J=7.43 Hz, 3H); ¹³C NMR (126 MHz, CDCl3) δ 171.39 (s), 164.66 (s), 160.49 (d, ¹J_{CF}=252.08 Hz), 136.41 (s), 134.72 (d, ³J_{CF}=7.91 Hz), 132.19 (s), 131.60 (d, ³J_{CF}=10.68 Hz), 130.13 (s), 127.16 (d, ²J_{CF}=13.28 Hz), 124.45 (d, ⁴J_{CF}=3.53 Hz), 122.84 (d, ³J_{CF}=3.62 Hz), 116.57 (s), 116.33 (d, ²J_{CF}=22.06 Hz), 64.85 (s), 24.24 (s), 10.60 (q, J=10.72 Hz); ¹⁹F NMR (471 MHz, CDCl₃) δ −112.58; HRMS (ESI/Q-TOF): m/z [M+H]⁺ calcd for C₁₇H₁₄BrFN₂O: 361.03463; found: 361.03605; HPLC Purity: 96.46%.

### Example 20. Synthesis of ethyl 8-bromo-4-ethyl-6-(2-fluorophenyl)-4H-benzo[f]imidazo[1,5-a][1,4]diazepine-3-carboxylate (36)

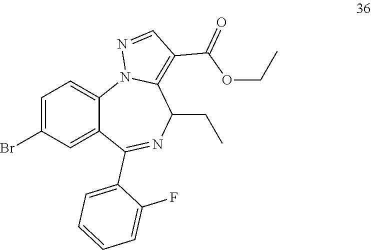 [SMILES: CCOC(=O)c1cnn2c1C(CC)N=C(c1ccccc1F)c1cc(Br)ccc1-2]

A three stopper RB flask was purged with nitrogen and vacuum 3×. Compound 35 (1.02 g, 2.83 mmol) was dissolved in anhydrous tetrahydrofuran (10.7 mL) and added to the reaction flask. The mixture was cooled to −20° C. using a dry ice/IPA bath. A solution of 1M potassium tert-butoxide in anhydrous tetrahydrofuran (3.68 mL) was added dropwise over the course of 10 min, at which time the reaction color turned to a deep orange. Upon completion of the addition, the mixture was allowed to stir at −20° C. for 40 min. Diethyl chlorophosphate (0.57 mL, 3.96 mmol) was added dropwise over the course of 5 min while maintaining a temperature of −20° C. After 3.5 hr, no more conversion was observed via TLC (100% EtOAc). Isocyanoacetate (0.40 mL, 3.68 mmol) was added dropwise over the course of 5 min followed by the addition of a solution of 1M potassium tert-butoxide in anhydrous tetrahydrofuran (3.68 mL) at −20° C. The reaction was then warmed to room temperature for 2 hr at which point all of the intermediate had been consumed via TLC (100% EtOAc). The reaction was then quenched with 5% aqueous sodium bicarbonate (40 mL), and the product was extracted with ethyl acetate (40 mL). The organic layer was washed with 10% aqueous sodium bicarbonate (40 mL) followed by 20% aqueous NaCl (mL). The organic layer was then dried with MgSO4 and then concentrated under reduced pressure. The resulting residue was triturated with a 50% mixture of tert-butyl methyl ether in hexanes (15 mL) at 55° C. for 20 hr. The tert-butyl methyl ether/hexanes mixture was decanted, and the solid product was slurried in 100% hexanes (20 mL) at 55° C. for 4 hr. The desired product was collected by filtration to yield an off-white solid (755.27 mg, 58.5%): ¹H NMR (500 MHz, CDCl3) δ 7.90 (s, 1H), 7.67-7.65 (dd, J=3.60, 2.25 Hz, 1H), 7.54-7.51 (m, 1H), 7.44-7.38 (m, 2H), 7.34-7.33 (d, J=2.10 Hz, 1H), 7.22-7.19 (dt, J=3.19, 0.85 Hz, 1H), 7.01-6.98 (m, 1H), 6.44-6.40 (q, J=5.43 Hz, 1H), 4.41-4.24 (m, 2H), 1.70-1.61 (m, 1H), 1.53-1.44 (m, 1H), 1.36-1.33 (t, J=7.15, 3H), 0.86-0.83 (t, J=7.37 Hz, 3H); ¹³C NMR (126 MHz, CDCl3) −20° C. S 161.83 (s), 161.72 (s), 158.78 (d, ¹J_{CF}=250.77 Hz), 139.27 (s), 133.80 (d, ³J_{CF}=3.77 Hz), 132.28 (s), 131.80 (s), 131.20 (d, ³J_{CF}=8.28 Hz), 130.06 (d, ⁴J_{CF}=2.01 Hz), 129.82 (s), 129.42 (s), 127.06 (d, ²J_{CF}=12.35 Hz), 123.58 (d, ³J_{CF}=3.36 Hz), 122.6 (s), 119.91 (s), 115.23 (d, ²J_{CF}=21.23 Hz), 59.86 (s), 55.43 (s), 21.40 (s), 13.36 (s), 10.45 (s); ¹⁹F NMR (471 MHz, CDCl3) −20° C. δ −111.73, −111.96; HRMS (ESI/Q-TOF): m/z [M+H]⁺ calcd for C₂₂H₁₉BrFN₃O₂: 456.07174; found: 456.07166; HPLC Purity: 99.85%.

### Example 21. Synthesis of 8-bromo-4-ethyl-6-(2-fluorophenyl)-4H-benzo[f]imidazo[1,5-a][1,4]diazepine-3-carboxylic acid (37)

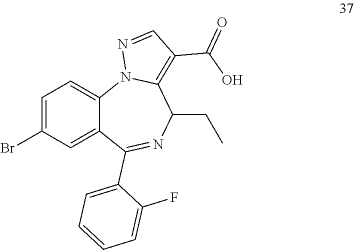 [SMILES: CCC1N=C(c2ccccc2F)c2cc(Br)ccc2-n2ncc(C(=O)O)c21]

Compound 36 (248.31 mg, 0.54 mmol) was dissolved in tetrahydrofuran (17.5 mL) and cooled to 0° C. Solid sodium hydroxide was added (652.9 mg, 16.32 mmol), followed by the addition of H₂O (410 L). The reaction was then removed from the ice bath and gently heated to 80° C. for 18 hr. When all of the starting material had dropped to the baseline via TLC (100% EtOAc), the reaction was cooled to room temperature. Acetic acid was added until the pH was observed to be ˜5, and the reaction was then warmed to 50° C. and allowed to stir for 20 hr. The reaction was then concentrated to dryness under reduced pressure. The residue was dissolved in H₂O (4.5 mL) and portioned into 0.5 mL fractions. To each fraction was added an additional 1 mL of H₂O causing the desired product to precipitate out of solution. The fractions were centrifuged and the solution was decanted. The solid fractions were combined and washed with an additional 7 mL of H₂O to remove any residual acetic acid. The product was then collected by filtration to yield a white powder. The solid was slurried in 5 mL of 5 M HCl at 95° C. for 18 hr upon which the diazepine ring had been completely broken by TLC (1.8 mL MeOH+0.1 mL AcOH+4.3 mL chloroform). The mixture was then cooled to room temperature and the product was collected by filtration. The solid material was then washed with hot IPA (1 mL, 2×) and the product was again filtered to yield the hydrochloride salt of the open form of the title compound, 1-(1-(4-bromo-2-(2-fluorobenzoyl)phenyl)-4-carboxy-1H-imidazol-5-yl)propan-1-aminium chloride. (189.23 g, 81.2%): ¹H NMR (500 MHz, MeOD) δ 8.32 (s, 1H), 7.89-7.87 (m, 1H), 7.81-7.79 (d, J=8.65 Hz, 1H), 7.59-7.55 (m, 2H), 7.38 (m, 1H), 7.35-7.32 (dd, J=3.20, 0.90 Hz, 1H), 7.19-7.15 (m, 1H), 6.49-6.45 (d, J=8.13 Hz, 1H), 1.80-1.72 (m, 1H), 1.64-1.55 (m, 1H), 0.95-0.92 (t, J=7.35 Hz, 3H); ¹³C NMR (126 MHz, MeOD) δ 164.74 (s), 164.06 (s), 160.02 (d, ¹J_{CF}=249.14 Hz), 139.33 (s), 135.76 (d, ²J_{CF}=28.40 Hz), 135.42 (d, ⁴J_{CF}=2.21 Hz), 135.16 (s), 133.78 (s), 132.72 (d, ³J_{CF}=17.25 Hz), 131.07 (s), 130.82 (s), 128.10 (d, ³J_{CF}=12.87 Hz), 124.55 (s), 124.42 (s), 124.30 (s), 121.77 (s), 120.57 (s), 115.74 (d, 2J_{CF}=21.04 Hz), 56.43 (d, J=39.09 Hz), 22.18 (t, J=33.97 Hz), 10.24 (d, J=7.56 Hz); ¹⁹F NMR (471 MHz, MeOD) δ −115.63; HRMS (ESI/Q-TOF): m/z [M+H]⁺ calcd for C₂₀H₁₅BrFN₃O₂: 428.04044; found: 428.04032; HPLC Purity: 95.76%.

For the closed form 7-bromo-3-ethyl-5-(2-fluorophenyl)-1,3-dihydro-2H-benzo[e][1,4]diazepin-2-one: ¹H NMR (500 MHz, d₆-DMSO) δ 8.26 (s, 1H), 8.17-8.14 (dt, J=3.25, 1.44 Hz, 1H), 7.64-7.59 (m, 2H), 7.40-7.37 (dt, J=3.22, 1.11 Hz, 1H), 7.18-7.17 (m, 1H), 6.58-6.57 (d, J=1.68 Hz, 1H), 3.11-3.04 (m, 1H), 2.65-2.57 (m, 1H), 0.87-0.84 (t, J=7.31 Hz, 1H), 0.71-0.68 (t, J=7.28 Hz, 3H); ¹³C NMR (126 MHz, d₆-DMSO) δ 164.38 (s), 159.34 (s), 139.02 (s), 135.24 (s), 133.38 (s), 132.33 (s), 132.15 (s), 131.90 (s), 131.64 (s), 129.48 (d, ³J_{CF}=24.41 Hz), 128.93 (s), 128.71 (s), 126.23 (s), 124.18 (s), 121.78 (s), 115.07 (s), 114.88 (s), 57.52 (s), 22.19 (s), 9.77 (s).

### Example 22. Determination of GABA(A) Receptor Binding

GABAA receptor binding: Rat brain membranes were prepared from frozen tissue that was thawed on ice and homogenized on ice in 10 volumes of cold lysis buffer (50 mM Tris HCl, pH 7.4, containing protease inhibitor cocktail from Roche) using a Polytron homogenizer (6 pulses and 10 seconds per pulse). The homogenate was centrifuged at 1,000×g for 10 min at 4° C. to obtain the supernatant. The supernatant was then centrifuged at 40,000×g for 20 min, and the resulting supernatant decanted and replaced with the same ice-cold lysis buffer. Two or three additional rounds of homogenization-centrifugation were performed to ensure thorough homogenization and also to wash out endogenous ligands. The final pellet was resuspended in the same buffer and homogenized one last time. The rat brain suspension was diluted in buffer (50 mM Tris HCl, 2.5 mM CaCl2, pH 7.4), followed by the addition of [3H]-flunitrazepam (0.6-4.0 nM in DMSO) and test compounds in DMSO at different concentrations to reach a final of volume of 125 μL per well. Total binding and nonspecific binding were determined with reference compound clonazepam. In brief, plates are usually incubated at room temperature and in the dark for 90 min. Reactions are stopped by vacuum filtration onto 0.3% polyethyleneimine (PEI) soaked 96-well filter mats using a 96-well Filtermate harvester, followed by three washes with cold PBS buffer. Scintillation cocktail is then melted onto the microwave-dried filters on a hot plate and radioactivity counted in a Microbeta counter. The data (n=6) were analyzed by nonlinear regression.

### Example 23. Determination of Aqueous Solubility

5-50 mg of compound was added to 500 μL of PBS buffer at pH 7.4. The pH was adjusted if necessary, using a 1M NaOH solution. The solutions were vortexed for 10 s, sonicated for 2 min and agitated with a horizontal shaker in a closed vial for 24 h. The mixtures were transferred to an Eppendorf tube and centrifuged for 5 min at 16000×g followed by filtration through 0.22 m cellulose acetate spin X centrifuge filter (Costar). 200 μL of filtrate was transferred to a new Eppendorf tube and 200 μL of methanol was added and then subsequent dilution was made in 50:50 methanol/PBS buffer water if necessary. After mixing, 50 μL were transferred into a 384 well plate (Coring UV star, 781801) for UV detection at 250-600 nm (Tecan M1000). The assay was carried out with three independent samples of each compound. The concentration of each solution was determined with a calibration curve in 50:50 methanol/PBS buffer water. Absorbance of corresponding methanol PBS blank were recorded and subtracted from the absorbance of calibration curve solutions and from the samples.

### Example 24. Determination of Permeability

The artificial membrane was prepared by carefully pipetting 15 μL of the 5% (v/v) hexadecane in hexane solution to each of the wells of the donor plate. The plate was placed into a fume hood for 1 h to ensure complete evaporation of the hexane. After the hexane had evaporated, 300 μL of PBS with 5% (v/v) DMSO was added to each of the wells of the acceptor plate. The hexadecane treated donor plate was then placed on top of the acceptor plate taking care that the underside of the membrane is completely in contact with the solution in each of the acceptor wells. 300 μM solutions were prepared of each compound in 5% (v/v) DMSO in PBS and 150 μM was transferred in triplicate to the donor wells. The lid was placed on the plates and the entire plate sandwich was placed into a closed humid environment. The container was then placed on a reciprocal shaker for agitation at about 100 rpm. The time at the beginning of the incubation was recorded, as this is a thermodynamic-based assay. The incubation was then allowed to continue for 18 h. The donor plate was removed and 50 μL of the acceptor solution was transferred to the UV plate. Drug solutions at the theoretical equilibrium concentration (100 μM) was also prepared and transferred to the UV plate. The absorbance of the solutions in the UV plate was then scanned from 250-600 nm with 1 nm steps and a 5 nm bandwidth. Log Pₑ was calculated as follows (Equation 1).

$$log Pe = log \{ C \times - ln ( 1 - \frac{{[ Drug ]}_{A}}{{[ Drug ]}_{E}} ) \} \qquad Equation 1$$

$$Where C = ( \frac{{V}_{A} \times {V}_{D}}{( {V}_{D} + {V}_{A} ) A \times T} )$$

The relative permeability (cm/s) of the small molecules was calculated with Equation 1, where V_{D} is the volume of the donor well in cm³ (150 L), V_{A} is the volume in the acceptor well in cm³ (300 μL), A is the active surface area of the membrane in cm² (0.283 cm²), T is the incubation time of the assay in seconds, [Drug]_{A} is the absorbance of the compound in the acceptor well after the incubation period, and [Drug]_{E} is the absorbance of the compound at the concentration of the theoretical equilibrium (as if the donor and acceptor solutions were simply combined).

### Example 25. Determination of Cytotoxicity of Compounds

Compounds with increased cytotoxicity can be identified using an in vitro cytotoxicity assay. Therefore, human embryonic kidney 293T (HEK293T) cell lines were purchased (ATCC) and cultured in 75 cm2 flasks (CellStar). Cells were grown in DMEM/High Glucose (Hyclone, #SH3024301) media to which non-essential amino acids (Hyclone, #SH30238.01), 10 mM HEPES (Hyclone, #SH302237.01), 5×106 units of penicillin and streptomycin (Hyclone, #SV30010), and 10% of heat inactivated fetal bovine serum (Gibco, #10082147) were added. Cells were harvested using 0.05% Trypsin (Hyclone, #SH3023601). The cell viability assay was evaluated using CellTiter-Glo™ Luminescent Cell Viability Assay Kit (Promega, Madison, WI), which contains luciferase and all its substrate except ATP. The controls for the cytotoxicity assay used were (E)-10-(bromotriphenylphosphoranyl)decyl 4-(4-(tert-butyl)phenyl)-4-oxobut-2-enoate (400 μM in DMSO, positive control) and DMSO (negative control). All luminescence readings were performed on a Tecan Infinite M1000 plate reader. Small volume transfers were performed on the Tecan Freedom EVO liquid handling system with a 100 nL pin tool transfer (V&P Scientific). Serial dilutions were done in 96-well polypropylene plates (Coming, #3365) and assays were conducted in 384-well white optical bottom plates. The assays were carried out in quadruplet in three independent runs. The data were normalized to the controls and if possible analyzed by nonlinear regression (GraphPad Prism).

### Example 26. GABA_{A} Receptor Affinity, Cytotoxicity, Solubility, and Permeability of Compounds

TABLE 1
GABA_{A} receptor affinity determined by competition
assay with [³H]-flunitrazepam and rat brain.

| | % Inhibition at 10 | |
|---|---|---|
| Compound | μM | IC₅₀ (nM) |
| 20 | n.d. | n.d. |
| 21 | 0 | 0 |
| 24 | n.d. | n.d. |
| 25 | 31.4 | n.d. |
| 26 | 97.0 | 9.1 |
| 27 | 59.4 | 1833 |
| 28 | 23.8 | n.d. |
| 29 | 1.9 | n.d. |
| 30 | n.d. | n.d. |
| 31 | 47.8 | n.d. |
| 32 | n.d. | n.d. |
| 33 | 4.7 | n.d. |
| 34 | 74.4 | 4053 |

TABLE 2
Cytotoxicity of compounds determined in the presence
of human kidney cells. None of the investigated
compounds induced cytotoxicity at 400 μM.

| Compound | LD₅₀ HEK293 (μM) |
|---|---|
| 20 | >400 |
| 21 | >400 |
| 22 | >400 |
| 23 | >400 |
| 24 | >400 |
| 25 | >400 |
| 26 | >400 |
| 27 | >400 |
| 28 | >400 |
| 29 | >400 |
| 30 | >400 |
| 31 | >400 |
| 32 | >400 |
| 33 | >400 |
| 34 | >400 |

TABLE 3
GABA_{A} Receptor Affinity, Cytotoxicity, Solubility, and Permeability

| | Aqueous | Permeability^{c}(log Pₑ | Cytotoxicity^{d} | GABA_{A}R binding, % |
|---|---|---|---|---|
| Comp | solubility^{a} (μM) | (cm/s) | LD₅₀ (μM) | inhibition at 10 μM (IC₅₀) |
| 8 | 319 ± 24 | −5.7 ± 0.01 | >150 | 70 (1246) |
| 35 | 397 ± 31 | −5.7 ± 0.04 | >150 | 95 (134) |
| 2 | 321 ± 19 | −5.6 ± 0.05 | >150 | 97 (42) |
| 3 | 109 ± 16 | −5.5 ± 0.16 | >150 | 71 (2035) |
| 4 | 98 ± 4 | −6.8 ± 0.27 | >120 | 13 (n.d.) |
| 5 | 94 ± 7 | −7.1 ± 0.09 | 87 ± 7 | 33 (n.d.) |
| 6 | 39 ± 3 | −5.2 ± 0.15 | >150 | 90 (665) |
| 15 | 103 ± 5 | −6.0 ± 0.12 | >150 | 78 (733) |
| 36 | 417 ± 17 | −5.7 ± 0.01 | >300 | 61 (175) |
| 9 | 141 ± 20 | −5.1 ± 0.10 | >120 | 94 (87) |
| 10 | 186 ± 17 | −5.2 ± 0.03 | >120 | 83 (859) |
| 11 | 144 ± 7 | −5.8 ± 0.10 | 106 ± 9 | 19 (n.d.) |
| 12 | 141 ± 19 | −6.0 ± 0.06 | 96 ± 11 | 74 (6222) |
| 13 | 54 ± 2 | −5.6 ± 0.04 | >150 | 34 (n.d.) |
| 18 | 53 ± 3 | −4.6 ± 0.03 | >150 | 56 (509) |
| 22 | 96 ± 1 | −4.9 ± 0.01 | >120 | 22 (n.d.) |
| 34 | 4.4 ± 0.1 ^{b} | −7.0 ± 0.22 | >300 | 74 (4053) |
| 37 | 77.6 ± 0.3 ^{b} | −6.6 ± 0.10 | >300 | 102 (145) |
| 26 | 8.2 ± 0.1 ^{b} | −6.8 ± 0.09 | >300 | 97 (9) |
| 27 | 58.7 ± 4.4 ^{b} | −6.6 ± 0.10 | >300 | 59 (1833) |
| 28 | 46.7 ± 0.6 ^{b} | −6.7 ± 0.04 | >300 | 2 (n.d.) |
| 29 | 46.9 ± 1.3 ^{b} | −6.6 ± 0.10 | >300 | 24 (n.d.) |
| 30 | 36.2 ± 0.4 ^{b} | −6.6 ± 0.03 | >300 | 18 (n.d.) |
| 20 | 40.3 ± 0.5 ^{b} | −6.7 ± 0.12 | >300 | 55 (59) |
| 24 | 12.8 ± 0.1 ^{b} | −6.4 ± 0.04 | >300 | 58 (1832) |
| 32 | 15.1 ± 0.1 ^{b} | −7.0 ± 0.14 | >300 | 74 (289) |

^{a}Shake flask, pH 7.2;
^{b} mM;
^{c}Permeability was measured using the parallel artificial membrane permeation assay at pH 7.4. Reference standards (log Pe): ranitidine (−7.0 cm/s) low permeability, naproxen (−5.0 cm/s) medium permeability, and verapamil (−4.0 cm/s) high permeability;
^{d}Cytotoxicity was determined using HEK293 cells using CellTiter-Glo;
^{e}Competition assay of GABA_{A}R ligand ³H-flunitrazepam using rat brain extract.

### Example 27. Rotarod Assay, to Determine Induced CNS Effects by Compounds

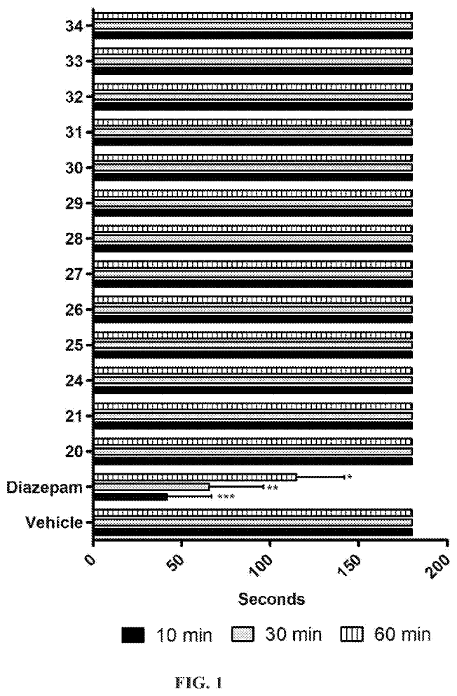

Compounds with the ability to cross the blood brain barrier and induce CNS effects modulating the sensorimotor skills of mice can be identified with the rotarod. Female Swiss Webster mice were trained to maintain balance at a constant speed of 15 rpm on the rotarod apparatus (Omnitech Electronics Inc., Nova Scotia, Canada) until mice could perform for three minutes at three consecutive time points. Separate groups of mice received intraperitoneal (i.p.) injections of compounds in vehicle (10% DMSO, 40% propylene glycol and 50% PBS) or oral gavage (p.o.) in vehicle (2% hydroxypropyl methylcellulose and 2.5% polyethylene glycol) in an approximate volume of 100 μL. Ten, thirty, and sixty minutes after each injection, mice were placed on the rotarod for three minutes. In case that mice fell from therotarod prior to 3 minutes the time was noted and averaged for the group of mice. An unpaired t-test (GraphPad Prism) was used to determine significance for *p<0.05, **p<0.01, and ***p<0.001. FIG. 1 shows the effect of compounds on sensorimotor coordination. Swiss Webster mice were tested on a rotarod at 15 rpm for 3 minutes at 10, 30, and 60 min following compound exposure. Mice (N=10) received a single injection (i.p. or p.o.) of test compound. The time of fall was recorded if it occurred prior to 3 minutes. Data are expressed as mean±SEM (N=10). Student's t-test was used to calculate significance: *(p<0.05), **(p<0.01) or ***(p<0.001) significance compared to vehicle-treated mice. None of the investigated compounds induced any sensorimotor impairments at the doses tested.

### Example 28. Relaxation of Guinea Pig Airway Smooth Muscle by Compounds

Adult male Hartley guinea pigs were euthanized by intraperitoneal pentobarbital (100 mg/kg). The tracheas were surgically removed and transected into cross-sections containing two cartilaginous rings. The rings are washed for one hour with at least five buffer exchanges to remove any pentobarbital. After the epithelium was removed with a cotton swab, the rings were suspended from two silk threads in a 4 mL jacketed organ bath (Radnoti Glass Technology), with one thread attached to a Grass FT03 force transducer (Grass-Telefactor) coupled to a computer via Biopac hardware and Acknowledge 7.3.3 software (Biopac Systems) for continuous digital recording of muscle tension. The rings were bathed in 4 ml of KH buffer solution (composition in mM: 118 NaCl, 5.6 KCl, 0.5 CaCh, 0.2 MgSO₄, 25 NaHCO₃, 1.3 NaH₂PO₄. 5.6 D-glucose) with 10 μM indomethacin (DMSO vehicle final concentration of 0.01%), which was continuously bubbled with 95% 02 and 5% CO₂ at pH 7.4, 37° C. The rings were equilibrated at 1 g of isotonic tension for 1 hour with new KH buffer added every 15 minutes. All rings were precontracted with 10 μM N-vanillylnonanamide (capsaicin analog) and then two cycles of cumulatively increasing concentrations of acetylcholine (0.1-100 μM) with extensive buffer washes between and after those two cycles with resetting of the resting tension to 1.0 g. Tetrodotoxin (1 μM) and pyrilamine (10 μM) were added to the buffer in the baths to eliminate the confounding effects of airway nerves and histamine receptors. After a stable baseline at 1.0 gresting tension was established, tracheal rings were contracted with 1 μM of substance P. After the peak contraction was reached, indicated concentrations of compounds or vehicle (0.1% DMSO) was added to the bath. The percentage of initial contraction remaining at indicated time points after compound exposure was expressed as a percentage of the remaining contractile force in vehicle-treated tissues and compared between groups.

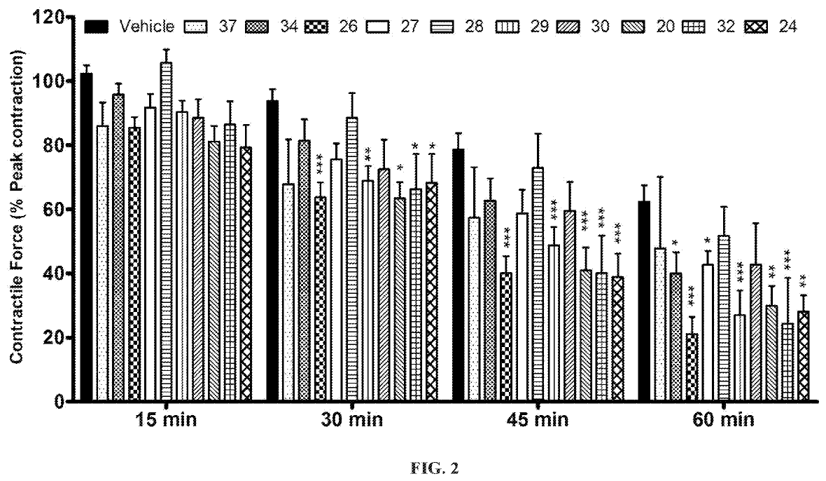

FIG. 2. shows airway smooth muscle contractile force in guinea pig tracheal rings. Tracheal rings were contracted with 1 mM substance P and then treated with 50 mM of compounds or vehicle control (0.1% DMSO). The percent of remaining contractile force was measured at various time points and expressed as a percent of the initial substance P induced contractile force. (N>6) A 2way ANOVA was used to calculate significance with *(p<0.05), **(p<0.01) or ***(p<0.001) p-values are given for each condition. FIG. 2:

### Example 29. Determination of Reduction of Airway Hyperresponsiveness Using a Predisposed Mouse Strain (A/J mice)

Measurement of sRaw using the Buxco FinePointe Non-Invasive Airway Mechanics instrument (DSI, St. Paul, MN). Mice were trained once a day for five days to become accustomed to the measuring chambers during nebulization and data acquisition. Instrument calibration was carried out before each experiment. Specific airway resistance (sRaw) was computed with FinePoint software using ventilation parameters recorded for the nasal and thoracic chambers. Compounds were nebulized as indicated for each experiment. Methacholine was dissolved in PBS and nebulized as indicated for each experiment. Nebulizers were calibrated for each measurement. Usually, nebulization occurred for one minute followed by a three-minute data acquisition and a one minute pause before the next methacholine nebulization. Data analysis was carried out with GraphPad Prism (GraphPad, San Diego, CA) using 2-way ANOVA and Bonferroni post test.

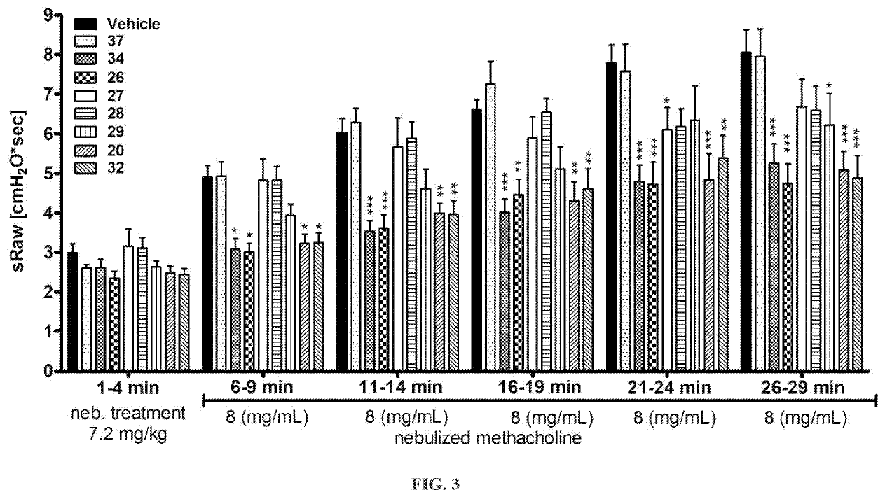

sRaw was measured with female A/J mice that received nebulized vehicle or treatment (7.2 mg/kg) followed by repeated treatment with nebulized methacholine. Data are depicted in FIG. 3 as means±SEM of n=10. * and *** indicate p<0.05 and p<0.001 significance between vehicle and drug treated animals using a 2-way ANOVA with Bonferroni correlation.

### Example 30. Virus-Induced Asthma Model

Efficacy of GABA_{A}R compounds in chronic lung disease will be studied in a virus induced asthma model. C57BL/6 mice (at 6-20 weeks of age; groups of 10) will be inoculated intranasally with 2×105 pfu SeV (Strain 52; ATCC) or UV-inactivated SeV (UV-SeV) (69), at day 0. Mice are monitored daily for weight and activity; with chronic disease being well established by day 49 post-inoculation (P-1). Four experimental groups will be arranged, with test compound (or vehicle) administered i.p. during days 49-56 P-1. For prophylactic studies, treatment would be administered during days 13-21, followed by disease measurement at days 49-56; to model a dosing regimen that corresponds to childhood treatment following RSV exposure (thus, investigating if immune modulation early after viral infection or during the acute post-infection phase can influence development of later inflammatory lung disease). Serial non-invasive AHR measurements (sRAW) in response to methacholine will be measured in all Groups on days 13, 49, and 56 PI. Following AHR measurements on day 56, animals will be euthanized and bronchoalveolar lavage fluid (BALF), blood, and tissue samples will obtained. BALF will be collected in 1 mL PBS, centrifuged, and the cell supernatants collected for cytokine analysis as above. The cell pellet will be resuspended in Roswell Park Memorial Institute (RPMI) medium and samples taken for differential cell counts (Diff-Quik) and flow cytometry. For flow cytometry, cell preparations will be stained with fluorophore labeled monoclonal antibodies to mouse CDld (Invitrogen) and Mac-3 (BD) for M2 macrophages or CD3 and NKI. 1 (both Invitrogen) for NKT cells. Antibody labeled cells will be examined with a FACS Calibur instrument (BD Biosciences) and data analyzed with FlowJo software (Tree Star, Ashland, OR) (69, 70, 84). All other lung tissue and biochemical testing will be performed and data analyzed as in the OVA model.

### Example 31. Mouse Atopic Dermatitis Model

An established mouse model of atopic dermatitis (AD) was used to evaluate the efficacy of PI-301 ((R)-8-bromo-6-(2-fluorophenyl)-4-methyl-4H-benzo[f]-imidazo[1,5-a][1,4]diazepine-3-carboxylic acid) in reducing ear thickness, scratching frequency, and inflammatory markers following MC903 challenge (for background on the model see: J Invest Dermatol 138, 2606-2616; Proc Natl Acad Sci USA, 103, 11736-11741; Sci Transl Med, 5, 170ra116; J Invest Dermatol, 138, 1555-1563). The increase of ear thickness is based on the increased diameter of stratum, epidermis, and dermis as determined by standard histological analysis. The thickening of the stratum is based on parakeratosis and hyperkeratosis, the thickening of the epidermis is based on acanthosis and spongiosis and the thickening of the dermis is based on inflammatory infiltration. This reflects increased numbers of neutrophils, mast cells, and, to a lesser extent, eosinophils.

C57/BL6 male mice 8 weeks of age were group housed under a 12 h light/dark cycle. For oral dosing, groups of 8 mice were each given twice daily treatment by voluntary feeding of vehicle (peanut butter) or PI-301 (100 mg/kg in vehicle). Animal weights were recorded daily. Three times per week animals were administered MC903 (1 nmol) on both ears topically. Ear thickness was measured with calipers and photographed.

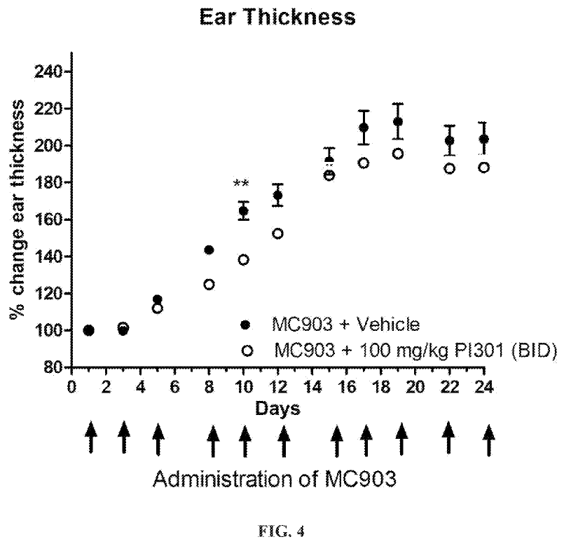

As shown in FIG. 4, oral administration of PI-301 reduced the increase of ear thickness due to MC903 application (application days shown as arrows on x-axis). Significant differences were observed on day 10. Overall ear thicknesses were lower with PI-301 compared to vehicle treated animals.

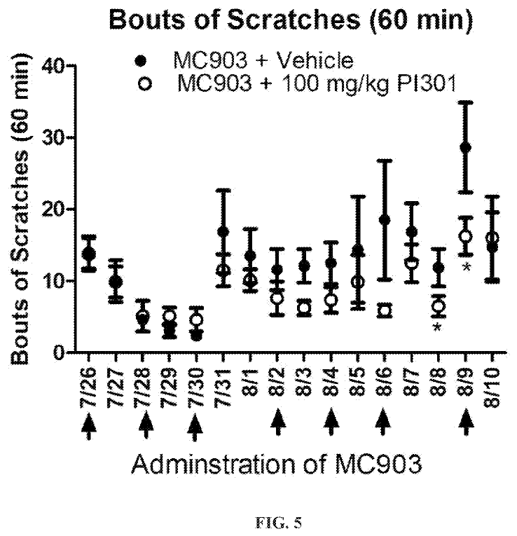

As shown in FIG. 5, the number of scratches was significantly reduced on days 19 and 22. Overall, the treated groups were lower when increased scratching was observed in the vehicle treated group.

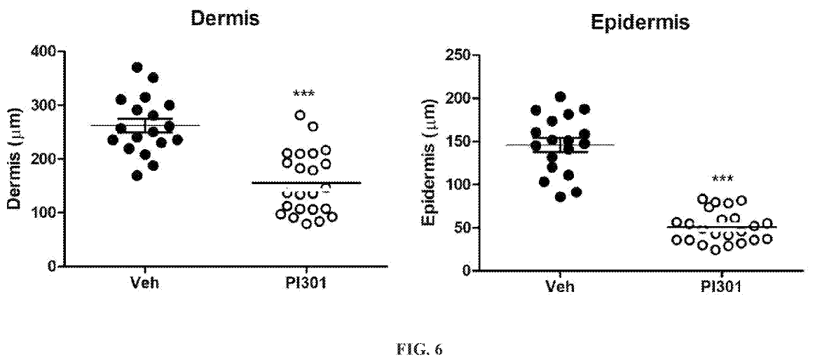

As shown in FIG. 6, significant differences (p<0.005) were observed between the thicknesses of the dermis and epidermis between mice treated with PI-301 compared to vehicle (veh) treatment (as determined by histological measurement of hematoxylin and eosin-stained ear sections).

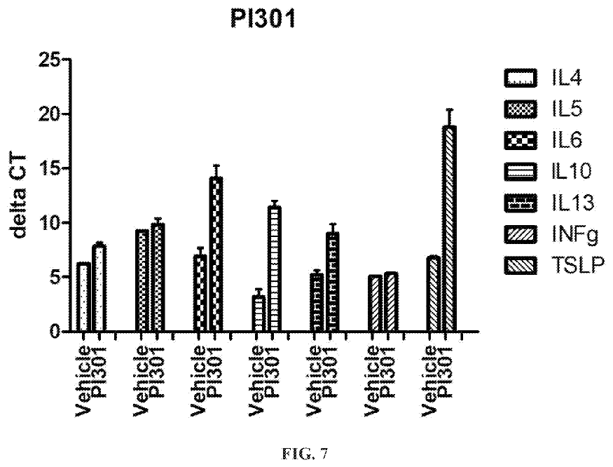

RT-PCR was used to assay the change in cytokines in MC903 challenged animals treated with PI-301. Left mouse ears stored at −20° C. were thawed, rinsed with water and homogenized in 500 μL RLT buffer (Qiagen, 79216). The liquid was added to a QIAshredder (Qiagen, 79654) and centrifuged at 6000 g for 2 min. 350 μL of the supernatant was combined with 350 μL 70% ethanol and mRNA purified with a RNeasy Mini Kit (Qiagen, 74104). mRNA was analyzed using the Accuris qMAX Green One-Step RT-qPCR Kit (Accuris Instruments) along with primers for IL-4, IL-5, IL-6, IL-10, IL-13, INFγ, and TSLP. Data were acquired using an Eppendorf Mastercycler Pro (45° C. 10 min, 95° C. 2 min, [95° C. 30 s, 55° C. 1 min, 72° C. 2 min]×45) and analyzed by the Delta and Delta-Delta Ct method. Each measurement was carried out in triplicate. IL-6, IL-10, IL-13 and TSLP showed reduced induction in mice challenged with MC903 and treated with PI301 compared to vehicle treatment (FIG. 7).

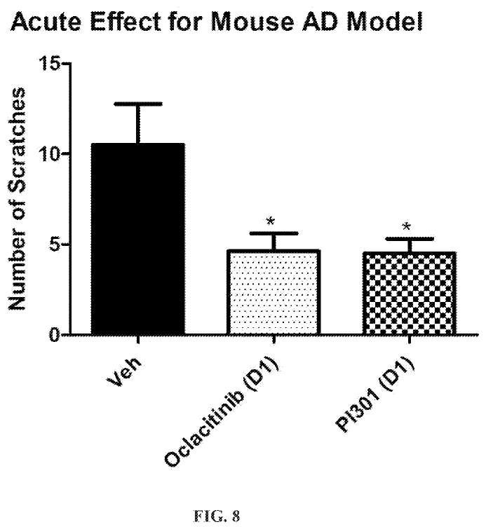

In another study, the acute treatment effects of oclacitinib (a JAK inhibitor approved for veterinary use) and PI-301 were studied in the MC903 model using C₅₇/BL6 mice. As described previously, MC903 was applied topically on both sides of both ears (1 nmol). On day 19 of the study (D1), drugs or vehicle were administered to individual treatment groups by oral gavage: oclacitinib (45 mg/kg) or PI-301 (100 mg/kg). Mice were video recorded for 60 minutes beginning 40 minutes after drug administration and scratches counted. The results show significant (p<0.05) reduction in scratches in both treatment groups compared to vehicle control (FIG. 8).

### Example 32. Chronic Obstructive Pulmonary Disease (COPD) Model

Efficacy of GABA_{A}R compounds will be studied by lipopolysaccharide (LPS) lung challenge in mice as a model of human COPD. LPS is a proinflammatory stimulant that is present as a contaminant in cigarette smoke, air pollution, and organic dusts. In humans, chronic exposure to LPS-laden dusts results in decreased lung function. In the acute model, LPS induces a mixed inflammatory reaction with increases in neutrophils and increased tumor necrosis factor (TNF), IL-1, and other mediators in bronchoalveolar fluid. Prior to administration, each test compound is diluted in a buffer solution vehicle (phosphate buffered saline, pH 7.4) and filter sterilized. Test compound is administered i.p. in a total volume of 100 μL, in each of the indicated days (thus 3 doses). Approximately one hour after the final i.p. compound administration, mice receive LPS intratracheally using a non-surgical procedure. Mice are first anesthetized by subcutaneous (s.c.) injection with ketamine hydrochloride and xylazine hydrochloride solution (Cat. no. Kl 13; Sigma; 50 mg/kg ketamine HCl). LPS (Cat. no. L2880; Sigma, type O55:B5), dissolved in 50 μL sterile 0.9% NaCl, is instilled intratracheally (i.t.) (20 g LPS/mouse) via a cannula and syringe (2×25 μL), followed by 100 μL air. Sham-treated mice are instilled i.t. with 50 μL sterile 0.9% NaCl. After i.t. treatment, mice are kept in an upright position for 10 minutes to allow the fluid to spread throughout the lungs. Mice are allowed to recover from anesthesia and the sacrificed 24 hours after using cervical CO₂ asphyxiation. Blood is collected via heart puncture in EDTA-containing tubes, immediately centrifuged (2000×g, 10 minutes, 4° C.) and plasma was stored at −80° C. Lungs tissue is removed and snap-frozen for RNA-isolation and MPO analysis. For immunohistochemical analyses lung tissue is placed in 10% phosphate-buffered formalin (pH 7.4).

### Example 33. Immune Arthritis Model

Groups of 8-10 male DBA/1J mice (Jackson Laboratories, Bar Harbor, ME, USA) 8-10 weeks of age are immunized with 200 mg bovine collagen II (bCII, Chondrex, Redmond, WA, USA) in 50% complete Freund's adjuvant intradermally at the base of the tail. Mice are similarly boosted 21 days later with 100 mg of bCII in incomplete Freund's adjuvant. Groups of control mice are treated with sham immunizations without bCII. Food and water consumption, body weights, as well as clinically observable joint inflammation are measured throughout the treatment course. Beginning after the initial immunization, mice are administered GABAA receptor agents of the instant invention 3 times per week for 8 weeks. Compound dosing will be determined as described in Example 27. Serum collagen-specific IgG, IgG1, and IgG2a antibodies in individual control and experimental mice 8 weeks after the final immunization are characterized by microtiter plate ELISA In the ELISA bCII is used as antigen for coating the plate wells and isotype-specific, fluorophore-conjugated rabbit anti-mouse antibodies are used to quantify primary antibody binding. Wells in the ELISA plates are read using a standard fluorescent plate reader. Compound efficacy will be evidenced by reduction of clinical joint inflammation and/or reduction in IgG antibody titers to bCII in the treated in comparison to control mice.

### Example 34. Autoimmune Diabetes Model

Non-obese diabetic (NOD) mice have been used for 30 years in the study of diabetes. NOD mice are characterized by insulitis, a leukocytic infiltrate of the pancreatic islets. Decreases in pancreatic insulin content occur spontaneously in females at about 12 weeks of age and several weeks later in males. Diabetic mice are hypoinsulinemic and hyperglucagonemic, indicating a selective destruction of pancreatic islet beta cells. Compounds of the instant invention are dosed QD or BID for 28 days via one of the routes of administration (PO, IP, IM, SC); control animals are similarly given vehicle doses. Doses are determined as described in Example 27. 8-10 mice are assigned per group. Animals are monitored twice per week for body weight, food consumption, water intake, and blood or urine glucose, are measured. Urine glucose can be determined using commonly available test strips (Bayer Diastix). Efficacy of compounds is evidenced by reduction in one or more of these clinical markers in the treated versus the control groups.

### Example 35. Overactive Bladder Model

C57BL/6 female mice (12 weeks, Harlan UK, Ltd) are euthanized by cervical dislocation. The bladder is removed after a midline laparotomy, the neck and trigone region cut away and the dome laid out as a sheet after an anterior wall incision. Strips with intact mucosa (5-7 mm length, 1-2 mm diameter) are dissected and tied in a horizontal trough between a fixed hook and an isometric force transducer. Preparations are superfused (3 ml·min-1) at 36° C. with Tyrode's solution. Test compound or DMSO vehicle are added to the superfusate and their effects on tension measured.

Unstimulated preparations are allowed to equilibrate under tension (20 mN) until a stable base-line is recorded. The preparation is exposed to 10 μM carbachol and peak and steady-state tension recorded. In the continued presence of carbachol the preparation is also exposed to test compound in cumulatively increasing concentrations (e.g., 1, 3, 10, 130, 100 μM) until a new steady-state is achieved at each concentration. For vehicle controls DMSO is added in rising concentrations (e.g., 0.01, 0.03, 0.1, 0.3, 1.0% (v/v)) after a steady-state contraction is achieved with carbachol.

Data are mean±SD; differences between multiple data sets were tested with repeated measures two-way ANOVA and Tukey post hoc tests.

All patents, publications and references cited herein are hereby fully incorporated by reference. In case of conflict between the present disclosure and incorporated patents, publications and references, the present disclosure should control.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. A compound of formula (I), or a pharmaceutically acceptable salt thereof, 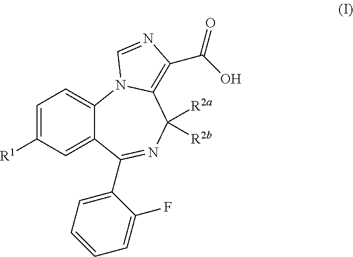
wherein
R¹ is H, halogen, CF₃, —OC₁₋₄alkyl, —C≡CH, or cyclopropyl; and
R^{2a} and R^{2b} are independently C₁₋₄alkyl, CF₃, or CCl₃; or R² and R^{2b} together with the carbon to which each attaches form a 3- to 6-membered saturated carbocyclic ring, the carbocyclic ring being optionally substituted with 1-4 substituents independently selected from the group consisting of methyl and fluoro.

2. The compound of claim 1, or a pharmaceutically acceptable salt thereof, wherein R^{2a} and R^{2b} are independently C₁₋₄alkyl, CF₃, or CCl₃.

3. The compound of claim 2, or a pharmaceutically acceptable salt thereof, wherein R^{2a} and R^{2b} are independently C₁₋₄alkyl.

4. The compound of claim 3, or a pharmaceutically acceptable salt thereof, wherein R^{2a} and R^{2b} are methyl.

5. The compound of claim 1, or a pharmaceutically acceptable salt thereof, wherein R^{2a} and R^{2b} together with the carbon to which each attaches form the optionally substituted 3- to 6-membered saturated carbocyclic ring.

6. The compound of claim 1 or 5, or a pharmaceutically acceptable salt thereof, wherein R^{2a} and R^{2b} together with the carbon to which each attaches form a cyclopropyl.

7. The compound of claim 1 or 5, or a pharmaceutically acceptable salt thereof, wherein R^{2a} and R^{2b} together with the carbon to which each attaches form a cyclobutyl.

8. The compound of claim 1 or 5, or a pharmaceutically acceptable salt thereof, wherein R^{2a} and R^{2b} together with the carbon to which each attaches form a cyclopentyl.

9. The compound of claim 1 or 5, or a pharmaceutically acceptable salt thereof, wherein R^{2a} and R^{2b} together with the carbon to which each attaches form a cyclohexyl.

10. The compound of any of claims 1-9, or a pharmaceutically acceptable salt thereof, wherein R^{b} is H.

11. The compound of any of claims 1-9, or a pharmaceutically acceptable salt thereof, wherein R¹ is Br.

12. The compound of any of claims 1-9, or a pharmaceutically acceptable salt thereof, wherein R¹ is —C≡CH.

13. The compound of any of claims 1-9, or a pharmaceutically acceptable salt thereof, wherein R¹ is cyclopropyl.

14. The compound of claim 1, or a pharmaceutically acceptable salt thereof, wherein the compound is selected from the group consisting of:

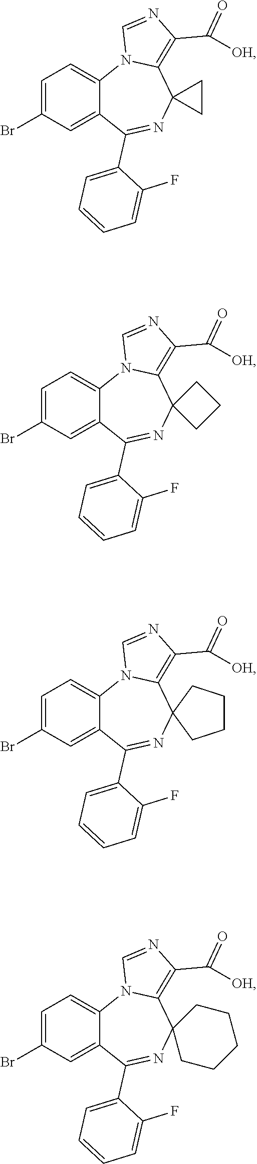 [SMILES: O=C(O)c1ncn2c1C1(CC1)N=C(c1ccccc1F)c1cc(Br)ccc1-2 ; O=C(O)c1ncn2c1C1(CCC1)N=C(c1ccccc1F)c1cc(Br)ccc1-2 ; O=C(O)c1ncn2c1C1(CCCC1)N=C(c1ccccc1F)c1cc(Br)ccc1-2 ; O=C(O)c1ncn2c1C1(CCCCC1)N=C(c1ccccc1F)c1cc(Br)ccc1-2]

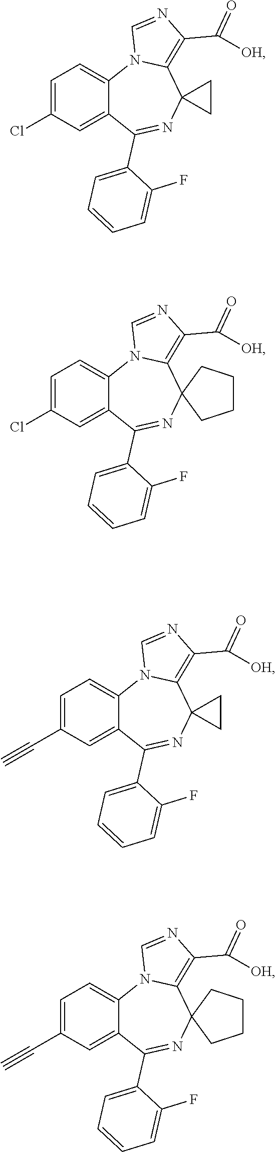 [SMILES: O=C(O)c1ncn2c1C1(CC1)N=C(c1ccccc1F)c1cc(Cl)ccc1-2 ; O=C(O)c1ncn2c1C1(CCCC1)N=C(c1ccccc1F)c1cc(Cl)ccc1-2 ; C#Cc1ccc2c(c1)C(c1ccccc1F)=NC1(CC1)c1c(C(=O)O)ncn1-2 ; C#Cc1ccc2c(c1)C(c1ccccc1F)=NC1(CCCC1)c1c(C(=O)O)ncn1-2]

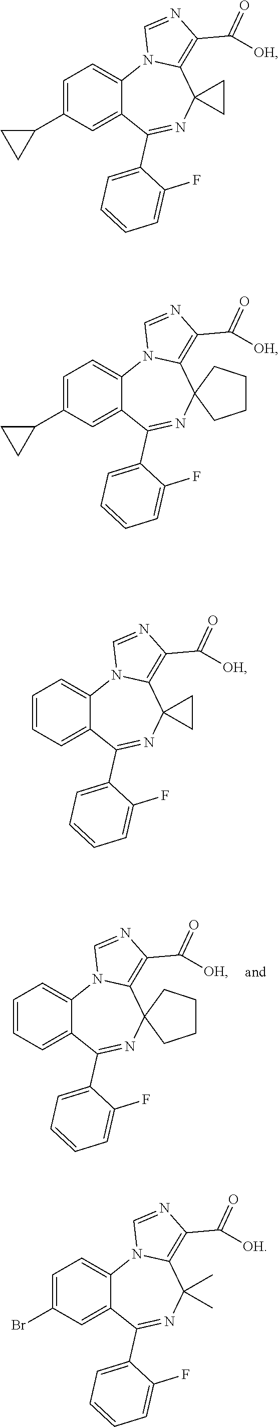 [SMILES: O=C(O)c1ncn2c1C1(CC1)N=C(c1ccccc1F)c1cc(C3CC3)ccc1-2 ; O=C(O)c1ncn2c1C1(CCCC1)N=C(c1ccccc1F)c1cc(C3CC3)ccc1-2 ; O=C(O)c1ncn2c1C1(CC1)N=C(c1ccccc1F)c1ccccc1-2 ; O=C(O)c1ncn2c1C1(CCCC1)N=C(c1ccccc1F)c1ccccc1-2 ; "and" ; CC1(C)N=C(c2ccccc2F)c2cc(Br)ccc2-n2cnc(C(=O)O)c21]

15. A pharmaceutical composition comprising a compound of any of claims 1-14, or a pharmaceutically acceptable salt thereof, and a pharmaceutically acceptable carrier.

16. A method of reducing inflammation in a subject suffering an inflammatory disease or disorder comprising administering to the subject, an amount of a compound of formula (II), or a pharmaceutically acceptable salt or composition thereof, that is effective to reduce inflammation in the subject, 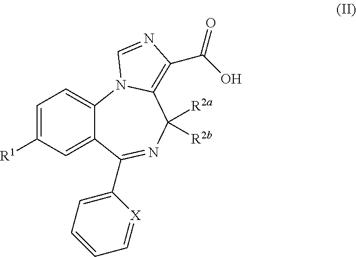
wherein
X is C—R^{x} or N;
R^{x} is halogen;
R¹ is H, halogen, CF₃, —OC₁₋₄alkyl, —C≡CH, or cyclopropyl; and
R^{2a} and R^{2b} are independently H, C₁₋₄alkyl, CF₃, or CCl₃; or R² and R^{2b} together with the carbon to which each attaches form a 3- to 6-membered saturated carbocyclic ring, the carbocyclic ring being optionally substituted with 1-4 substituents independently selected from the group consisting of methyl and fluoro.

17. The method of claim 16, wherein the inflammatory disease or disorder is an acute inflammatory disease or disorder.

18. The method of claim 16, wherein the inflammatory disease or disorder is a chronic inflammatory disease or disorder.

19. The method of claim 16, wherein the inflammatory disease or disorder is a respiratory disease or condition.

20. The method of claim 19, wherein the respiratory disease or condition is asthma, chronic obstructive pulmonary disease, bronchitis, emphysema, inflammatory diseases of the upper respiratory tract such as allergic rhinitis and allergic sinusitis, acute lung injury, acute respiratory distress syndrome (ARDS), lung infection, interstitial lung disease, or constrictive bronchiolitis.

21. The method of claim 16, wherein the inflammatory disease or disorder is a digestive tract disease or condition.

22. The method of claim 21, wherein the disease or disorder is an inflammatory bowel disease, Crohn's disease, ulcerative colitis, esophagitis, irritable bowel syndrome, celiac disease, gastritis, pancreatitis, proctitis, hepatitis, diverticulitis, or tropical sprue.

23. The method of claim 16, wherein the inflammatory disease or disorder is a dermatologic disease or condition.

24. The method of claim 23, wherein the disease or disorder is dermatitis, atopic dermatitis, rash, pruritis, eczema, acne, dandruff, cellulitis, psoriasis, rosacea, hives, shingles, lupus erythematosus, lichen planus, dermatitides, vasculitis, or bullous diseases.

25. The method of claim 16, wherein the inflammatory disease or disorder is septic shock, sepsis, systemic inflammatory response syndrome (SIRS), hemorrhagic shock, shock states induced by cytokine therapy (interleukin-2, tumor necrosis factor, immune checkpoint inhibition), organ transplantation and transplant rejection, head trauma, or inflammatory eye conditions such as uveitis, glaucoma and conjunctivitis.

26. The method of claim 16, wherein the inflammatory disease or disorder is: an arthritic disorder such as rheumatoid arthritis, ankylosing spondylitis, osteoarthritis or gouty arthritis; a heart disorder such as cardiomyopathy or myocarditis; atherosclerosis; neurogenic inflammation; diabetes; glomerulonephritis; a urological disorder such as overactive bladder and cystitis; Parkinson's disease; Huntington's induced dementias; amyotrophic lateral sclerosis (ALS); multiple sclerosis; necrotizing vasculitides such as polyarteritis nodosa; serum sickness; Wegener's granulomatosis; Kawasaki's syndrome; headaches such as migraine, chronic tension headaches, cluster or vascular headaches; or myocardial and cerebral ischemia/reperfusion injury.

27. A method of reducing airway constriction comprising administering to a subject in need thereof, an amount of a compound of formula (II), as defined in claim 16, or a pharmaceutically acceptable salt thereof, that is effective in reducing airway constriction in the subject.

28. The method of any of claims 16-27, wherein the compound, or pharmaceutically acceptable salt thereof is administered orally, topically, parenterally, or by inhalation.

29. The method of any of claims 16-28, wherein the subject is a human or non-human animal.

30. The method of any of claims 16-29, wherein:
X is C—R^{x} or N;
R^{x} is halogen;
R¹ is H, halogen, CF₃, —OC₁₋₄alkyl, —C≡CH, or cyclopropyl;
R^{2a} is C₁₋₄alkyl, CF₃, or CCl₃; and
R^{2b} is H, C₁₋₄alkyl, CF₃, or CCl₃.

31. The method of claim 30, wherein R¹ is halogen, —C≡CH, or cyclopropyl.

32. The method of claim 31, wherein R¹ is Cl, Br, or cyclopropyl.

33. The method of any of claims 30-32, wherein X is C—R^{x}.

34. The method of any of claims 30-32, wherein X is N.

35. The method of any of claims 30-34, wherein R^{2a} is C₁₋₄alkyl.

36. The method of any of claims 30-35, wherein R^{2b} is H.

37. The method of any of claims 30-36, wherein the compound has (R) stereochemistry at R^{2a} and R^{2b}.

38. The method of any of claims 30-36, wherein the compound has (S) stereochemistry at R^{2a} and R^{2b}.

39. The method of claim 30, wherein the compound is selected from the group consisting of

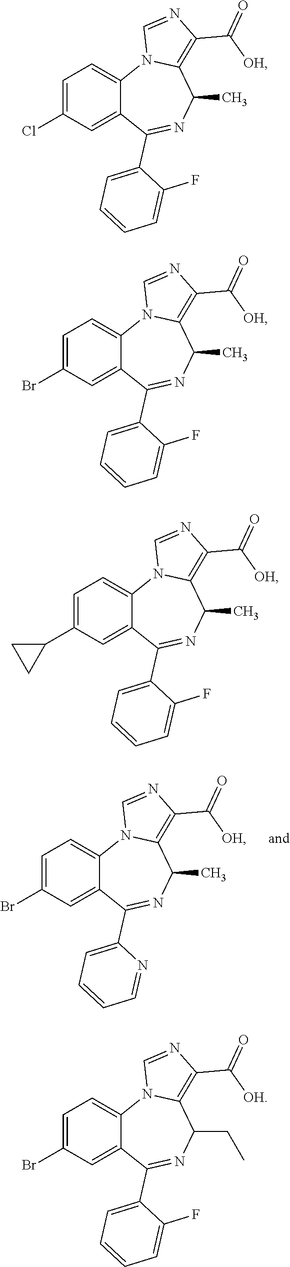

40. The method of any of claims 16-29, wherein the compound or its pharmaceutically acceptable salt or composition is as defined in any of claims 1-15.
